SEQUENCE LISTING (2 sequences)

SEQ ID NO: 1 - DNA, 20 bp, synthetic construct
  source 1..20: mol_type = other DNA
tccaagcaggctgttgctta

SEQ ID NO: 2 - DNA, 20 bp, synthetic construct
  source 1..20: mol_type = other DNA
agcaggcgagagtgtaggta

(19) United States
(12) Patent Application Publication
(10) Pub. No.: US 2025/0339587 A1
(43) Pub. Date: Nov. 6, 2025

(54) MESENCHYMAL STEM CELL-DERIVED EXTRACELLULAR VESICLE COMPOSITIONS AND USES THEREOF

(71) Applicants: Regents of the University of Minnesota, Minneapolis, MN (US); THE UNITED STATES GOVERNMENT AS REPRESENTED BY THE DEPARTMENT OF VETERANS AFFAIRS, Washington, DC (US)

(72) Inventors: Rosemary F. Kelly, Minneapolis, MN (US); Tammy Butterick, Washington, DC (US); Annie Shao, Minneapolis, MN (US)

(21) Appl. No.: 19/197,461

(22) Filed: May 2, 2025

Related U.S. Application Data
(60) Provisional application No. 63/642,145, filed on May 3, 2024.
(60) Provisional application No. 63/708,894, filed on Oct. 18, 2024.

(51) Int. Cl. A61L 27/38; A61L 27/24; A61L 27/54; A61L 27/56; A61L 27/58; C08L 89/04; C12N 5/077; C12N 5/0775

(52) U.S. Cl. CPC A61L 27/3834; A61L 27/24; A61L 27/54; A61L 27/56; A61L 27/58; C08L 89/04; C12N 5/0657; C12N 5/0662; A61L 2300/64; A61L 2430/20; C12N 2500/02; C12N 2502/1329; C12N 2533/54

(57) ABSTRACT

The present invention provides extracellular vesicle compositions. Also provided are methods of making an extracellular vesicle loaded scaffold using the extracellular vesicle compositions to increase revascularization. Methods of using the extracellular vesicle compositions and the extracellular vesicle loaded scaffold are provided. The scaffold may increase blood flow and oxygenation of the tissue thereby aiding in treatment of disease and injury.

### CROSS-REFERENCE TO RELATED APPLICATIONS

This application claims priority to U.S. Provisional Application Nos. 63/642,145 filed on May 3, 2024, and 63/708,894 filed on Oct. 18, 2024, the contents of which are incorporated by reference in their entireties.

### REFERENCE TO AN ELECTRONIC SEQUENCE LISTING

The contents of the electronic sequence listing (92017100641.xml; Size: 2,714 bytes; and Date of Creation: Apr. 28, 2025) is herein incorporated by reference in its entirety.

### BACKGROUND

Ischemic heart disease (IHD) affects over one hundred million patients. Although the mortality rate has decreased in recent years, it remains a leading cause of death worldwide^{1,2}. The IHD spectrum can present from acute myocardial infarction to chronic ischemia. Pre-clinical research of IHD most often focuses on myocardial infarction, characterized by the presence of infarcted tissue that cannot be rescued. However, many patients have chronic ischemia with preserved myocardial viability that is amenable to treatment. Hibernating myocardium (HIB) is a clinical diagnosis defined as chronically ischemic myocardium with reduced blood flow and function that retains viability and variable contractile reserve³. As HIB tissue is viable, the standard treatment is coronary artery bypass graft (CABG). However, CABG often results in incomplete functional recovery possibly due to persistent myocardial maladapations including prooxidant and proinflammatory signaling^{4,5}. In a porcine model of HIB that is amenable to CABG, the inventors have shown that systolic and diastolic function only slightly improves with CABG, which indicates a therapeutic gap^{6,7}. Studies have shown significant dysregulation in the mitochondrial proteome in HIB, characterized by decreased expression of ETC complexes, mitochondrial fusion proteins, and regulators of mitochondrial biogenesis^{8,9}. These findings suggest that the disease mechanism centers around dysregulation of mitochondrial morphology, proteome, and mitochondrial function¹⁰. Inadequate mitochondrial bioenergetic capacity is present in HIB before and after CABG, as manifested by persistently reduced expression of ETC proteins⁹. This suggests that the process of mitochondrial dynamism is incomplete with CABG alone despite evidence that cardiac tissue is no longer ischemic following CABG⁵. Mitochondrial dysfunction that fails to fully recover despite revascularization may be an important therapeutic target for regenerative therapy. Accordingly, there remains a need in the art for novel therapies and methods of treating HIB.

### SUMMARY

The present invention provides methods for making an extracellular vesicle composition. In some embodiments, the method comprises (a) contacting a mesenchymal stem cell (MSC) in vitro under normoxic conditions with (i) conditioned media from an ischemic cell or (ii) a cell exposed to hypoxic culture conditions for at least 12 hours prior to indirect co-culture with the MSC, to produce a contacted MSC culture, and (b) collecting extracellular vesicles from the contacted MSC culture to generate the extracellular vesicle composition. In some embodiments, the conditioned media from the ischemic cell is generated by exposing a cell to hypoxic culture conditions for at least 12 hours and harvesting the culture media from the cells to create the conditioned media from the ischemic cell of step (a). In some embodiments, the contacting step of (a) is for at least 6 hours. In some embodiments, the method further comprises contacting a scaffold with the extracellular vesicle composition of step (b) to create an extracellular vesicle loaded scaffold. In some embodiments, the scaffold is made of an absorbable material and does not comprise intact cells. In some embodiments, the ischemic cell is a cardiomyocyte which has been exposed in vitro to hypoxic conditions for at least 12 hours, wherein the ischemic cardiomyocyte and the mesenchymal stem cell are indirectly co-cultured for at least 6 hours, and wherein the scaffold is a collagen sponge. In some embodiments, the hypoxic culture conditions comprise culturing cells in 0.5% to 10% oxygen for at least 12 hours.

Another aspect of the present disclosure provides a method of using the extracellular vesicle composition described herein or an extracellular vesicle loaded scaffold. In some embodiments, the extracellular vesicle composition or extracellular vesicle loaded scaffold is administered to a site of reduced blood supply in a subject. In some embodiments, the composition or scaffold provides sustained release of extracellular vesicles to a tissue or area of a subject to increase revascularization and air in the treatment of disease and injury. In some embodiments, the injury is an ischemic injury. In some embodiments, the subject is undergoing surgery, and the extracellular vesicle composition or extracellular vesicle scaffold is administered to the site of injury during surgery.

Another aspect of the present invention provides a method of adjuvant vascular bypass therapy comprising culturing cardiomyocytes in hypoxic conditions for at least 12 hours, switching the cardiomyocytes to normoxic conditions, and indirectly co-culturing mesenchymal stem cells (MSC) in the same culture for at least 12 hours, or contacting MSC with conditioned media from the hypoxic cardiomyocyte culture, collecting extracellular vesicles from the contacted MSC culture and applying the extracellular vesicles to the site of a bypass graft. In some embodiments, the extracellular vesicles are contacted to an absorbable scaffold to generate an extracellular vesicle loaded scaffold. In some embodiments the bypass therapy is cardiac bypass, cerebral bypass or peripheral vascular bypass.

### BRIEF DESCRIPTION OF THE DRAWINGS

The present technology can be better understood by reference to the following drawings. The drawings are merely exemplary to illustrate certain features that may be used singularly or in combination with other features and the present technology should not be limited to the embodiments shown.

FIG. 1: Schematic illustration of the isolation of Mesenchymal stem cells (MSCs)-derived exosomes and preparation of exosome embedded collagen patch to be sutured directly on anteroseptal region of porcine hibernating myocardium during coronary artery bypass graft (CABG) surgery. H&E, Hematoxylin and eosin.

FIG. 2. Experimental design to test epicardial placement of an exosome-embedded collagen patch (EXP) during coronary artery bypass grafting (CABG) in porcine hibernating myocardium (HIB) and its effects on cardiac function and fibrosis. Juvenile swine are implanted with a rigid plastic constrictor that creates a gradual, nonoccluding stenosis as the animal grows over 12 weeks. This creates a model of single-vessel HIB characterized by decreased blood flow and decreased cardiac function with preserved tissue viability. At 12 weeks, animals undergo left internal mammary artery-left anterior descending artery (LIMA-LAD) bypass surgery along with epicardial placement of an EXP in the anteroseptal region of the myocardium. Four weeks following this treatment, cardiac function is measured by cardiac magnetic resonance imaging and the animal is humanely killed for histological and trans-mission electron microscopy (TEM) analysis of the cardiac tissue. MRI, Magnetic resonance imaging; LV, left ventricle. Definitions and abbreviations for FIG. 2: Hibernating Myocardium (HIB): Decreased mitochondrial respiratory function, Dysregulated mitochondrial morphology, Increased interstitial fibrosis, and Reduced cardiac function. Revascularization+Exosome patch (CABG+EXP): Recovery of mitochondrial respiratory function and morphology, Reduced interstitial fibrosis, and Improved cardiac function. Implications: Adjuvant EXP during CABG recovers cardiac function by improving mitochondrial respiratory function and reducing interstitial fibrosis allowing a recovering heart to better respond under stress. Future studies to focus on understanding the mechanisms underlying exosome-driven recovery of cardiac function to pave the way for future application of an “off”-the-shelf cell-free adjunctive therapy. CABG=Coronary artery bypass graft; EXP=Stem-cell derived exosome patch; HIB=Hibernating myocardium; LAD=left anterior descending artery; MRI=Magnetic resonance imaging; TEM=Transmission electron microscopy.

FIG. 3. Transwell coculture of mesenchymal stem cells (MSC) during reoxygenation restores mitochondrial respiratory function in hibernating myocardium (HIB) cardiomyocytes. (A), Measurement of oxygen consumption rate (OCR) in cardiomyocytes following transwell coculture with MSCs resulted in an overall increase in mitochondrial respiratory activity. Basal (B) and maximal respiration (C) were increased following coculture with MSCs (**P<0.01 and ****P<0.0001, respectively). (D), Adenosine triphosphate (ATP) production was increased following MSC coculture (****P<0.0001). (E), Quantitative polymerase chain reaction shows increased fold change in expression of Atp5f1a following coculture with MSCs (***P<0.001). (F), Measurement of nuclear-bound peroxisome proliferator-activated receptor gamma coactivator 1-alpha (PGC1a) protein expression was increased following MSC coculture (***P<0.001). Inhibition of MSC exosome release by GW4869 abrogated the response of hypoxic H9C2 cells to MSC coculture as measured by OCR (G), ATP production (H), basal respiration (I), and maximal respiration (J). (*P<0.05; **P<0.01; ***P<0.0001). One-way analysis of variance test was used for statistical analyses. Values are presented as mean±SEM. FCCP, Carbonyl cyanide-p-tri-fluoromethoxyphenylhydrazone; ns, nonsignificant FIG. 4. Differentially expressed micro RNAs (miRNAs) identified in mesenchymal stem cells-derived exosomes from coculture with hypoxic vs nor-moxic H9C2 cardiomyocytes. Target prediction tools miRDB, miRmap, and miRSystem were used to identify targets from a predefined list of genes of in-terest, including RELA (nuclear factor kappa-light-chain enhancer of activated B cells), INFG (interferon gamma), ATP5F1 (adenosine triphosphate synthase), IL1B (interleukin 1 beta), and PPARGC1A (peroxisome proliferator-activator receptor gamma coactivator 1-alpha).

FIG. 5. Magnetic resonance imaging (MRI) assessment of regional systolic function. Measurements of % wall thickening were done using cardiac MRI with and without dobutamine infusion at 5 mg/kg/minute. Systolic function, as measured by wall thickening %, was compared among 4 groups (normal, hibernating myocardium [HIB], coronary artery bypass grafting [CABG], and CABG b exosome). At rest, the wall thickening % measurements are similar among normal (n ¼ 6), HIB (n ¼ 11), CABG (n ¼ 12), and CABG b exosome (n ¼ 6) animal groups. However, there was a decrease in wall thickening % in the HIB compared with the normal group (P ¼ 0.02). In the CABG group, there was no statistical improvement compared with the HIB group (P ¼ 0.24). However, in the CABGb exosome group, there was an increase when compared with the HIB group (P ¼ 0.02). One-way analysis of variance test was used for statistical analyses. Values are presented as mean±SD.

FIG. 6. Magnetic resonance imaging (MRI) assessment of peak filling rates. Measurements of peak filling rate (PFR) were done using cardiac MRI with and without dobutamine infusion at 5 mg/kg/minute. Diastolic function, as measured by PFR/end diastolic volume (EDV), was compared among 4 groups (normal, hibernating myocardium [HIB], coronary artery bypass grafting [CABG], and CABG b exosome). At rest, the PFR/EDV measurements are similar among normal (n ¼ 6), HIB (n ¼ 17), CABG (n ¼ 19), and CABG b exosome (n ¼ 6) animal groups. However, there was a decrease in PFR/EDV in the HIB compared with the normal group (P<0.03). In CABG, there was a trend toward improvement compared with the HIB group (P ¼ 0.07). However, in the CABG b exosome group, there was an increase when compared with the HIB group (P<0.02). One-way analysis of variance test was used for statistical analyses. Values are presented as mean±SD.

FIG. 7. Histopathological assessment of inflammation and fibrosis. Left anterior descending artery (LAD) tissue was obtained from animals in all 4 groups and was qualitatively analyzed for presence of fibrosis and myofibroblasts. Trichrome stains (103) of the LAD region showed normal amount of interstitial fibrosis in normal (A), extensive presence of fibrosis in endomysium in hibernating myocardium (HIB) (B), focal extension of fibrosis in endomysium in coronary artery bypass grafting (CABG) (C), and (D) normal amount of fibrosis similar to normal controls in the CABG bexosome group. Presence of myofibroblasts was assessed using alpha-smooth muscle actin (SMA) stain (403). (E), In the normal group, there was an absence of alpha-SMA stain in the myocardial tissue beyond the expected smooth muscle in the blood vessels (white arrowhead). (F), The HIB group showed increased number of myofibroblasts between bundles of myofibers (outlined by arrows). (G), The CABG group showed very few myofibroblasts associated with alpha-SMA stain (outlined by arrows). (H), The CABG b exosome group showed minimal to no myofibroblasts (smooth muscle in vessel marked by white arrowhead). See Table 1 for scoring/grading of fibrosis and inflammation.

FIG. 8. Tissue samples from the left anterior descending (LAD) region underwent transmission electron microscopy (TEM) imaging for measurements of mitochondrial ultrastructure. Each group included 3 animals with a total of approximately 500 mitochondria measured in each group. Compared with normal, hibernating myocardium (HIB), and coronary artery bypass grafting (CABG) alone, CABG b exosome animals show an increase in mitochondrial area (A), perimeter (B), and aspect ratio (C) (****P<0.0001). LAD tissue from animals in 4 groups was analyzed for mitochondrial morphology. TEM images (88003 magnification) show mitochondrial size and alignment in normal myocardium (D), dysregulation of mitochondrial size and alignment in HIB (E), Treatment with CABG (F), and CABG b exosome patch (G) shows recovery of mitochondrial morphology. One-way analysis of variance test was used for statistical analyses. Values are presented as mean±SD.

FIG. 9. (A) Exosome number and size distribution from cell culture media isolated following H9C2 cardiomyocytes and MSC co-culturing was determined by a NanoSight NS300 configured with a 488 nm laser and a high sensitivity scientific CMOS camera. Replicate samples (n=4) were analyzed under constant flow conditions (flow rate=50 @25° C.), 15×60 s videos were captured, and data was analyzed using NTA 3.1.54 software. (B) In vitro cumulative release profile of exosome-laden collagen patch (EXP) shows sustained released of exosomes in PBS over 7 days. (C) MSC-derived exosomes loaded onto collagen sponge and stained with Hematoxylin and eosin (H&E) staining showing collagen matrix structure (D) Fluorescent imaging of EXP loaded with BODIPY-labeled exosomes. (E) Final patch consists of two collagen sponges embedded with exosomes placed directly onto anteroseptal region of myocardium with vicryl patches sutured over (marked by arrows).

FIG. 10: Cardiac magnetic resonance images of cardiac anatomy. (A) Left anterior descending artery stenosis (shown by white arrow) is present at 12 weeks following constrictor placement. (B) Left internal thoracic artery graft is patent (shown by white arrow) 1 month following coronary artery bypass graft surgery. LAD=Left anterior descending artery; LIMA=Left internal mammary artery; MRI=Magnetic resonance imaging

FIG. 11: Cardiac angiographic image showing LAD stenosis (white arrowhead) due to constrictor placement and patent LIMA-LAD bypass graft anastomosis.

### DETAILED DESCRIPTION

The present invention provides methods for making an extracellular vesicle composition and an extracellular vesicle loaded scaffold to provide sustained release of extracellular vesicles to a tissue or area of a subject. The composition and scaffold allows an extracellular vesicle suspension to be targeted to a specific area and release extracellular vesicles over time, thereby aiding in treatment of disease and injury.

One aspect of the present disclosure provides a method of making an extracellular vesicle composition. In some embodiments, the method comprises (a) contacting a mesenchymal stem cell (MSC) in vitro under normoxic conditions with (i) conditioned media from an ischemic cell or (ii) a cell exposed to hypoxic culture conditions for at least 12 hours prior to indirect co-culture with the MSC, to produce a contacted MSC culture, then (b) collecting extracellular vesicles from the contacted MSC culture to generate the extracellular vesicle composition. In some embodiments, the method further comprises contacting a scaffold with the extracellular vesicle composition of step (b) to create an extracellular vesicle loaded scaffold.

Mesenchymal stem cells are stromal cells with the ability to self-renew and also exhibit multilineage differentiation. MSCs can be isolated from a variety of tissues, such as umbilical cord, endometrial polyps, menses blood, bone marrow and adipose tissue. Mesenchymal stem cells can differentiate into a variety of cell types, including bone cells (osteoblasts), cartilage cells (chondrocytes), muscle cells (myocytes) and fat cells that give rise to marrow adipose tissue (adipocytes). The MSCs of the present invention may be isolated from a subject. MSCs of the present invention may also be derived or reprogramed from another cell, or be of human origin or non-human origin, for example porcine MSCs.

As used herein an ischemic cell is one which has been exposed to low or decreased oxygen for some period of time. An ischemic cell can be generated using hypoxic tissue culture conditions. Normal oxygen conditions are also sometimes called normoxia. Normoxia is considered to be normal levels of oxygen with regard to the physiological responses of living organisms and hypoxia is when oxygen is deficient for aerobic organisms. Normoxia is typically considered to be about 19%, 20% or 21% oxygen in cell culture. Tissue normoxia may be lower than tissue culture normoxia. In some embodiments, a cell is exposed to hypoxic cell culture conditions for at least 12 hours. In some embodiments, hypoxic cell culture conditions can last 8, 10, 12, 16, 18, 20, 24, 26, 30, 36, 48 or more hours and any amount of time in-between. In some embodiments the hypoxic conditions may be in a range of about 0.5% oxygen to about 10% oxygen and any amount of oxygen in-between. The time and percentage of oxygen creating the hypoxic conditions can vary, for example 1% oxygen can be used for 24 hours, or 5% oxygen may be used for 48 hours. In some embodiments, a cell is exposed to hypoxic cell culture conditions of 1% for at 24 hours. In some embodiments, 1% oxygen for 24 hrs may be called mild hypoxic conditions. In some embodiments, a cell is placed in hypoxic conditions to generate an ischemic cell. In some embodiments, an ischemic cell exposed to hypoxic conditions may be switched into normoxic conditions, or conditions of standard, normal oxygen. In some embodiments, a mesenchymal stem cell may be co-cultured with an ischemic cell in normoxic conditions.

The ischemic cell used herein may be any cell type able to withstand hypoxic conditions and grown in culture. Examples of cells which may be exposed to hypoxic conditions of the present disclosure include, but are not limited to, cardiomyocytes, bronchial cells, pneumocytes, gastrointestinal cells, central nervous system cells, and endothelial cells.

In some embodiments, the ischemic cell is a cardiomyocyte. The cardiomyocyte may be derived from an induced pluripotent stem cell, a primary cell or may be immortalized, as in a cardiomyocyte cell line. The cardiomyocyte may be exposed to hypoxic conditions, for example 1% oxygen for 24 hours to generate an ischemic cardiomyocyte as used herein.

In some embodiments, a contacted MSC culture is produced. A contacted MSC culture can be produced by contacting an MSC with conditioned media from an ischemic cell. A contacted MSC culture can also be produced by indirectly co-culturing a MSC with an ischemic cell under normoxic conditions.

The mesenchymal stem cell may be co-cultured with an ischemic cell indirectly by use of a transwell insert or some other means of physically separating the two cell types in the same culture. Transwell co-culture is an indirect culture system where cells are physically separated into two different populations that allow communication only via secretory factors. For example, an ischemic cell may be in a tissue culture well and a transwell insert can be added into the well that contains the mesenchymal stem cells. The two cell populations can communicate with paracrine signaling, but do not physically contact each other. Paracrine signaling allows cells to communicate with each other by releasing signaling molecules that interact with surrounding cells. For example, an ischemic cell may release signaling molecules into the media which then contact the mesenchymal stem cell in the same culture. The media that contains these signals released from an ischemic cell may be called conditioned media. In some embodiments an MSC can be indirectly co-cultured with an ischemic cell to produce a contacted MSC culture. In some embodiments, the MSC is indirectly co-cultured for at least about 1 hour, 2 hours, 3 hours, 4 hours, 5 hours, 6 hours, 7 hours, 8 hours, 9 hours, 10 hours, 12 hours, 14 hours, 18 hours, 24 hours, 48 hours and any time in between. In some embodiments, the MSC is indirectly co-cultured for at least 6 hours to 24 hours with an ischemic cell.

As used herein, “conditioned media” contains biological components secreted by an ischemic cell. For example, proteins, lipids, nucleotides and extracellular vesicles, including exosomes, released by an ischemic cell in culture. In some embodiments an MSC can be contacted with the conditioned media from an ischemic cell to produce a contacted MSC culture. In some embodiments, the MSC is contacted with the conditioned media for at least about 1 hour, 2 hours, 3 hours, 4 hours, 5 hours, 6 hours, 7 hours, 8 hours, 9 hours, 10 hours, 12 hours, 14 hours, 18 hours, 24 hours, 48 hours and any time in between. In some embodiments, the MSC is contacted for at least 6 hours with conditioned media from an ischemic cell.

Extracellular vesicles are lipid bilayer-delimited particles that are naturally released from almost all types of cells but cannot replicate. Extracellular vesicles range in diameter from around 20-30 nanometers to as large as 10 microns or more. Extracellular vesicles can include exosomes, microvesicles and apoptotic bodies. Exosomes are a type of membrane bound extracellular vesicle that are produced in the endosomal compartment of most eukaryotic cells. Exosomes range in size from 30 to 150 nanometers. Exosomes may contain nucleic acids, proteins, lipids, and metabolites and mediate intercellular communication. In some embodiments, exosomes are collected from the mesenchymal stem cells after indirectly co-culturing the mesenchymal stem cell with an ischemic cell or conditioned media from an ischemic cell. In some embodiments, the extracellular vesicles comprise exosomes.

In some embodiments, extracellular vesicles are collected from the media of a contacted MSC culture to generate the extracellular vesicle composition described herein. In some embodiments, media from the contacted MSC culture is concentrated and lyophilized. Concentrated lyophilized media can be stored at room temperature. Concentrated lyophilized media can be reconstituted in saline and a specific concentration can be applied to a scaffold and used as described herein. In some embodiments, 1×10⁶ extracellular vesicles per ml, or 1×10⁷ extracellular vesicles per ml, or 1×10⁸ extracellular vesicles per ml, or 1×10⁹ extracellular vesicles per ml, or 1×10¹⁰ extracellular vesicles per ml or more, and any concentration in between are added to a scaffold.

In some embodiments, extracellular vesicles collected from the contacted MSC culture are contacted to an absorbable scaffold. An absorbable scaffold containing extracellular vesicles from the contacted MSC culture may also be called an extracellular vesicle loaded scaffold.

A scaffold, as described herein, can be used in the treatment of conditions and diseases and to aid in the repair and functional reconstruction of an injured area including vascularization and revascularization. In some embodiments a scaffold is contacted with the extracellular vesicle composition as described herein, an extracellular vesicle-contacted scaffold may also be called an extracellular vesicle-loaded scaffold. An extracellular vesicle loaded scaffold used herein does not contain any intact cells, such that the extracellular vesicle loaded scaffold is a cell-free therapy. A scaffold may be any sterile absorbable material that can contain the extracellular vesicle composition within it. As used herein “absorbable” means a material that once added inside or to the body, the material does not need to be removed from the body and will not be found in or on the body at a later time. An absorbable scaffold resolves within the body. An absorbable scaffold has the advantage of not requiring a second procedure to take it out, or having something left inside a body.

In some embodiments, the scaffold may be an absorbable hemostatic sponge. In some embodiments, the scaffold is an absorbable gelatin sponge. An absorbable gelatin sponge is a sterile hemostatic agent composed of purified gelatin. Without limitation, examples of absorbable gelatin sponges include GelFoam®, and Surgifoam®. In some embodiments, the scaffold may be an absorbable collagen sponge. Collagen sponges are made from sterile purified collagen. Without limitation, an example of a collagen sponge scaffold includes HeliSTAT®. The scaffold is extracellular vesicle-loaded, when the extracellular vesicle composition is contacted to the scaffold. In some embodiments, the scaffold does not contain whole or intact cells.

Another aspect of the present disclosure provides a method of using an extracellular vesicle composition or extracellular vesicle loaded scaffold described herein. In some embodiments, the extracellular vesicle composition or extracellular vesicle loaded scaffold is administered to a site of reduced blood supply in a subject in need thereof. In some embodiments, the extracellular vesicle loaded scaffold is held in place at the site of reduced blood supply by a surgical mesh. The surgical mesh maintains the position of the scaffold at the site of reduced blood supply. The surgical mesh may be of an absorbable material and may be a knitted or woven material. In some embodiments the surgical mesh may be adhered to the site by a physical means such as sutures, staples and/or adhesion. In some embodiments, the surgical mesh comprises polyglactin. By way of example, and not limitation, one type of surgical mesh may include Vicryl™.

In some embodiments, the extracellular vesicle composition or extracellular vesicle loaded scaffold described herein may be administered to a site of reduced blood supply in a subject in need. The term “subject” may be used interchangeably with the terms “individual” and “patient” and includes human and non-human mammalian subjects. A “subject in need thereof” as utilized herein may refer to a subject in need of treatment for a disease or disorder associated with reduced blood flow, or reduced oxygenation of a tissue. In some embodiments, the subject is in need of increased cardiac function. In some embodiments, the subject in need has an ischemic injury. Ischemic injury occurs when the blood supply to an area of tissue is cut off or reduced. Types of ischemic injury include, but are not limited to myocardial ischemia, mesenteric ischemia, peripheral ischemia, ischemic stroke, or transient ischemic attack. In some embodiments the subject in need is undergoing surgery. For example, a subject may be undergoing surgery for coronary heart disease, a wound, a burn, organ transplant, stroke, severed limb, or a bone fracture. A composition or scaffold described herein may be administered to the subject in need during the surgery. For example, a subject may be administered the composition or scaffold while undergoing coronary artery bypass surgery. In some embodiments, the composition or scaffold described herein may be administered to a cardiac muscle of a subject, wherein the scaffold increases cardiac function. In some embodiments, the ischemic cell and the mesenchymal stem cell may be from the same species as the species of the subject in need. For example, an ischemic human cardiomyocyte may be indirectly co-cultured with a human mesenchymal stem cell and the isolated extracellular vesicles created therefrom may be administered as part of a scaffold to a human subject. The cells used to generate the extracellular vesicles added to the scaffold may be allogeneic with the subject.

In some embodiments, a method of making an extracellular vesicle loaded scaffold to increase revascularization is provided. Increasing revascularization may also include vascularization. Vascularization is the process of growing blood vessels into a tissue to improve oxygen and nutrient supply. Revascularization is the restoration of perfusion to a body part or organ that is ischemia. In some embodiments, the extracellular vesicle-loaded scaffolds described herein aid in the revascularization to recover cardiac function. Without wishing to be bound by theory, the extracellular vesicle-loaded scaffolds may enhance mitochondrial function and reduce inflammation, which can aid revascularization. In particular, expression of mitochondrial mediators such as PGC-1α and mitochondrial proteins such as ATP synthase may be modulated, as well as mitochondrial area, perimeter and aspect ratio during revascularization. Expression of inflammatory mediators such as NFκB, IFNγ and IL-1β may also be modulated during revascularization. Revascularization following the methods described herein may also improve cardiac function.

As described herein, the inventors have shown that extracellular vesicles produced by contacted MSCs can be used therapeutically. Specifically, exosomes secreted from the contacted MSCs are shown to attenuate ischemic injury. Extracellular vesicles as described herein may contain any protein, nucleic acid, lipid or metabolite from the contacted MSC. For example, the inventors have shown the extracellular vesicles contain microRNAs (miRNA), for example miR-21-5p, miR-10a-5p, and miR-143-3p.

“Contacting” as used herein, e.g., as in “contacting a scaffold” or “contacting a site of reduced blood supply” refers to contacting a sample directly. For example, directly contacting extracellular vesicle to a scaffold, or directly contacting a scaffold to the site of injury or repair. Contacting may include the administration to a subject. “Contacting a mesenchymal stem cell” refers to contacting a cell or sample directly in vitro or ex vivo. Contacting encompasses administration to a solution, cell, tissue, mammal, subject, patient, or human. Further, contacting a cell includes adding an agent to a cell culture, for example adding conditioned media to a culture of mesenchymal stem cells. In some embodiments, a scaffold is contacted by extracellular vesicles such that the scaffold becomes an extracellular vesicle-loaded scaffold. In some embodiments, mesenchymal stem cells are contacted by conditioned media of an ischemic cell such that the ischemic cell is not in direct contact with the mesenchymal stem cell.

In some embodiments, an extracellular vesicle loaded scaffold described herein may be used as an adjunctive therapy, wherein the scaffold is used in addition to other medications, treatments and procedures to treat a subject in need. By way of example and not limitation, an extracellular vesicle loaded scaffold described herein may be administered during coronary artery bypass grafting. For example, a subject may undergo heart bypass surgery as part of a treatment for a blocked or narrowed artery, with the administration of a scaffold described herein as part of the treatment.

In some embodiments, the administration of an extracellular vesicle loaded scaffold described herein may aid in recovery of cardiac function. Cardiac function refers to the ability of the heart to efficiently pump blood throughout the body, which can be measured through a variety of metrics including volumetric measurements, myocardial strain, inward displacement, and hemodynamic forces.

In some embodiments, administration of an extracellular vesicle loaded scaffold described herein decreases inflammation and/or fibrosis. Inflammation is part of the process by which the immune system defends the body from harmful agents, such as bacteria and viruses. Inflammation can also be triggered by injury. Myocarditis is inflammation of the heart muscle (myocardium). Myocarditis can reduce the heart's ability to pump blood. Fibrosis is the development of fibrous connective tissue as a reparative response to injury or damage. Heart, cardiac or myocardia fibrosis refers to the excess deposition of extracellular matrix in the cardiac muscle and/or an abnormal thickening of the heart valves.

Another aspect of the present disclosure provides a method of adjuvant vascular bypass therapy. The method comprises culturing cardiomyocytes in hypoxic conditions or mild hypoxic conditions. Culturing in hypoxic conditions for about 24 hours was used in the Examples. Then the cardiomyocytes are switched to normoxic conditions, and indirectly co-cultured with mesenchymal stem cells in the same culture as the cardiomyocytes for at least 12 hours and up to 48 hours. A co-culture of about 24 hours was used in the Examples. Alternatively, media from cardiomyocytes grown in hypoxic conditions (conditioned media) may be added to mesenchymal stem cells in normoxic conditions. Extracellular vesicles are collected from the co-culture, or from the conditioned media exposed stem cell culture and applied to a scaffold to create an extracellular vesicle loaded scaffold which is applied to a site of a bypass graft. In some embodiments the hypoxic conditions comprise 0.5% to 10% oxygen, or 0.5% to 1.5% oxygen. In some embodiments the extracellular vesicles are contacted to an absorbable scaffold to generate an extracellular vesicle-loaded scaffold. In some embodiments, the extracellular vesicle-loaded scaffold is secured to the site of the bypass graft with a surgical mesh.

In some embodiments the bypass therapy is cardiac bypass, or cardiac bypass surgery. In some embodiments, the therapy is peripheral vascular bypass. Types of peripheral vascular bypass include aortobifemoral bypass, femoral-popliteal bypass, and femoral-tibial bypass. In some embodiments, the bypass is cerebral bypass.

### Additional Definitions

The present disclosure is not limited to the specific details of construction, arrangement of components, or method steps set forth herein. The compositions and methods disclosed herein are capable of being made, practiced, used, carried out and/or formed in various ways that will be apparent to one of skill in the art in light of the disclosure that follows. The phraseology and terminology used herein is for the purpose of description only and should not be regarded as limiting to the scope of the claims. Ordinal indicators, such as first, second, and third, as used in the description and the claims to refer to various structures or method steps, are not meant to be construed to indicate any specific structures or steps, or any particular order or configuration to such structures or steps.

All methods described herein can be performed in any suitable order unless otherwise indicated herein or otherwise clearly contradicted by context. The use of any and all examples, or exemplary language (e.g., “such as”) provided herein, is intended merely to facilitate the disclosure and does not imply any limitation on the scope of the disclosure unless otherwise claimed. No language in the specification, and no structures shown in the drawings, should be construed as indicating that any non-claimed element is essential to the practice of the disclosed subject matter.

Unless otherwise specified or indicated by context, the terms “a”, “an”, and “the” mean “one or more.” For example, “a molecule” should be interpreted to mean “one or more molecules.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus ≤10% of the particular term and “substantially” and “significantly” will mean plus or minus >10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion additional components other than the components recited in the claims. The term “consisting essentially of” should be interpreted to be partially closed and allowing the inclusion only of additional components that do not fundamentally alter the nature of the claimed subject matter.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure. Use of the word “about” to describe a particular recited amount or range of amounts is meant to indicate that values very near to the recited amount are included in that amount, such as values that could or naturally would be accounted for due to manufacturing tolerances, instrument and human error in forming measurements, and the like. All percentages referring to amounts are by weight unless indicated otherwise.

In those instances where a convention analogous to “at least one of A, B and C, etc.” is used, in general such a construction is intended in the sense of one having ordinary skill in the art would understand the convention (e.g., “a system having at least one of A, B and C” would include but not be limited to systems that have A alone, B alone, C alone, A and B together, A and C together, B and C together, and/or A, B, and C together). It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or ‘B or “A and B.”

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

The following examples are meant only to be illustrative and are not meant as limitations on the scope of the invention or of the appended claims.

### EXAMPLES

### Example 1

This study focuses on the ability of adjunctive cell-free therapy during revascularization to recover cardiac function through enhanced mitochondrial function and reduced inflammation.

### Methods

Preparation and Characterization of Exosomes from Porcine Mesenchymal Stem Cells (MSCs):

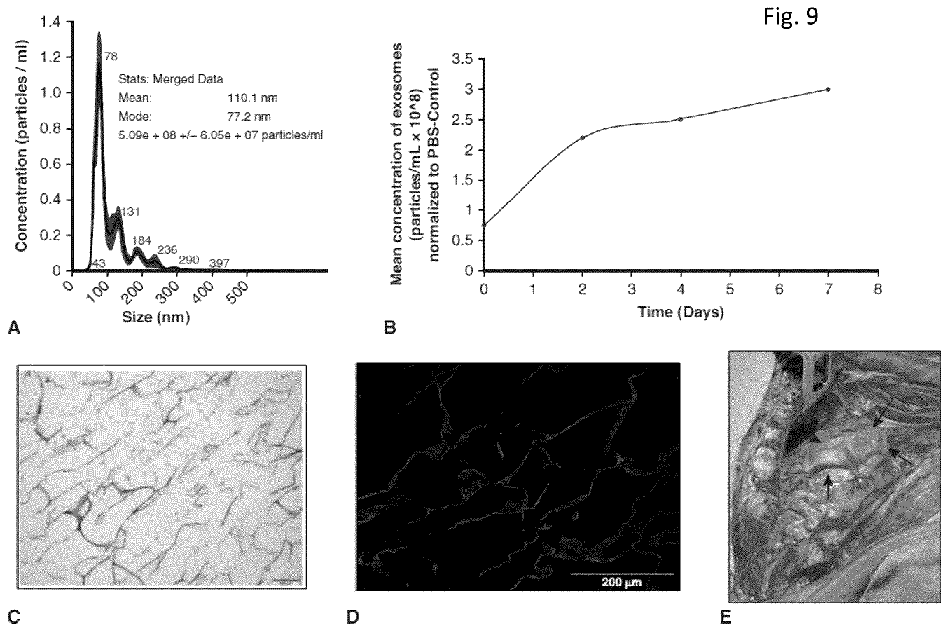

We isolated and validated bone-marrow-derived MSCs as described by Hocum Stone¹⁷. To simulate HIB, we exposed H9C2 cardiomyocytes to mild hypoxia (1% O₂ for 24 hours). H9C2 cells were moved to normoxic conditions for 24 hours (5% CO₂, 20% O₂, and 37° C.) and co-cultured with MSCs via transwell insert. Exosomes from co-cultured conditioned media were extracted using Invitrogen Total Exosome Isolation Reagent (Invitrogen, Gaithersburg, MD, United States) following manufacturer's instructions. Identification of exosomes was verified by western blot detection of common exosomal proteins with antibodies (Abcam, Cambridge, MA, USA) against CD-63 (1:1000). For exosome quantification and assessment of nanoparticle size and distribution, Nanoparticle Tracking Analysis (NTA) was performed (ZetaView, Particle Metrix, Meerbusch, Germany). Total protein (50 μg) of exosomes was dissolved in 500 μl of PBS, and the concentration and size distribution of exosomes samples were determined by a NanoSight NS 300 configured with a 488 nm laser and a high sensitivity scientific CMOS camera. Replicate samples (n=4) were analyzed under constant flow conditions (flow rate=50 @25° C.), 15×60 s videos were captured, and data were analyzed using NTA 3.1.54 software (FIG. 9A).

### Seahorse Assay of Mitochondrial Respiration and Function:

Mitochondrial respiration and ATP production were determined using Agilent Seahorse XF96e Analyzer (Seahorse Bioscience-Agilent, Santa Clara, CA, USA) 18 The primer sequences used forAtp5f1a are as follows Forward: 5′ TCCAAGCAGGCTGTTGCTTA 3′ (SEQ ID NO: 1) Reverse: 5′AGCAGGCGAGAGGTGTAGGTA 3′ (SEQ ID NO: 2). 96-well plates were seeded with 8000 H9C2 cells/well and exposed to hypoxic conditions for 24 hours, with or without subsequent MSC co-culture and/or 40 μm GW4869 (inhibitor of exosome release). MSC transwells were removed prior to measurement of oxygen consumption rate (OCR) in co-culture groups to ensure respiratory measurements reflected H9C2 respirations alone. A mitochondrial stress test to measure OCR was performed according to manufacturer recommendations.

Mitochondrial respiration and ATP production of H9C2 cardiomyocytes were determined using the Agilent Seahorse XF96e Analyzer (Seahorse Bioscience-Agilent, Santa Clara, CA, USA)¹⁸. 96-well plates were seeded with 8000 H9C2 cells per well and exposed to hypoxic conditions for 24 hours, with or without subsequent MSC co-culture and/or 40 μm GW4869 (inhibitor of exosome release). MSC transwells were removed prior to measurement of oxygen consumption rate (OCR) in the co-culture groups to ensure respiratory measurements reflected H9C2 respirations alone. A mitochondrial stress test to measure OCR was performed according to manufacturer recommendations. Briefly, growth medium was replaced with XF test medium (Eagles's modified Dulbecco's medium, 0 mM glucose, pH=7.4; Agilent Seahorse) supplemented with 1 mM pyruvate, 10 mM glucose, and 2 mM L-glutamine. Before the assay, the cells were incubated in a 37° C. incubator without CO₂ for 45 min to allow the pre-equilibration of the assay medium. The test was performed by first measuring the baseline OCR, followed by sequential OCR measurements after the injection of the following compound concentrations: 1p m oligomycin; 2 μm trifluoromethoxy carbonylcyanide phenylhydrazone (FCCP); 0.5p m rotenone/antimycin A. This allowed the measurement of the key parameters of the mitochondrial function, including the basal respiration, the ATP-linked respiration, the maximal respiration, the spare respiratory capacity, and the non-mitochondrial ATP production. Exosome release was inhibited as described previously^{E17}. Briefly, MSCs were cultured to 80% confluence in complete DMEM and incubated with 40 μm GW4869 diluted with 0.05% dimethyl sulfoxide (DMSO) (Sigma-Aldrich, D1692) for 24 hours; then, the medium was replaced with sEV-depleted FBS medium containing 40 μm GW4869, and the cells were incubated for another 48 hours before the medium was collected for exosome isolation. Control assessments were conducted after treatment with 0.05% DMSO solution.

### Total RNA Isolation and RNA-Sequence Analysis:

Exosomes were isolated from MSCs co-cultured with normoxic H9C2 cells and MSCs co-cultured with reoxygenated-hypoxic H9C2 cells as described above. Total RNA was isolated from the exosomes using standard protocol described in QIAGEN (QIAGEN GmbH, Hilden, Germany) ExoRNeasy Serum/Plasma Handbook.

Exosomes were isolated from MSCs co-cultured with normoxic H9C2 cells and MSCs co-cultured with reoxygenated-hypoxic H9C2 cells as described above. Total RNA was isolated from the exosomes using standard protocol described in QIAGEN (QIAGEN GmbH, Hilden, Germany) ExoRNeasy Serum/Plasma Handbook. Briefly, sample was mixed 1:1 with 2× binding buffer (XBP) and added to the exoEasy membrane affinity column to bind the extracellular vesicles to the membrane. After centrifugation, the flow-through was discarded and wash buffer (XWP) was added to column to wash off non-specifically retained material. After another centrifugation and discarding of the flow-through, the vesicles were lysed by adding QIAzol to the spin column, and the lysate was collected by centrifugation. The miRNeasy Serum/Plasma Spike-in Control (QIAGEN, Hilden) was added. Following addition of chloroform, thorough mixing, and centrifugation to separate organic and aqueous phases, the aqueous phase was recovered and mixed with ethanol. The sample-ethanol mixture was added to a RNeasy MinElute spin column and centrifuged. The column was washed once with buffer RWT, and then twice with buffer RPE followed by elution of RNA in water. This procedure allows concentrating the extracellular RNA from 4 ml plasma or serum into a final volume of 14 μl of water.

miRNA Profiling:

Preparation of RNA library and transcriptome sequencing was conducted by Novogene Co., LTD (Beijing, China). miRNA expression levels were estimated by TPM (transcript per million) as previously described¹⁹. Differential expression of two groups/conditions was performed using the DESeq R package (1.8.3). Gene ontology (GO) enrichment analysis was used on the target gene candidates of differentially expressed miRNAs. miRNA target analyses were performed using the miRDB and miRmap prediction tools, as well as the miRSystem database, which integrates seven prediction programs²⁰⁻²². Target predictions were cross-referenced with a list of HIB-specific inflammatory and metabolic genes of interest, including RELA (NF-κB), INFG (INF-γ), ATP5F1 (ATP synthase), IL1B (IL-1β), PPARGC1A (PGC-1α).

### Creation of MSC-Derived Exosome-Laden Collagen Patch (EXP):

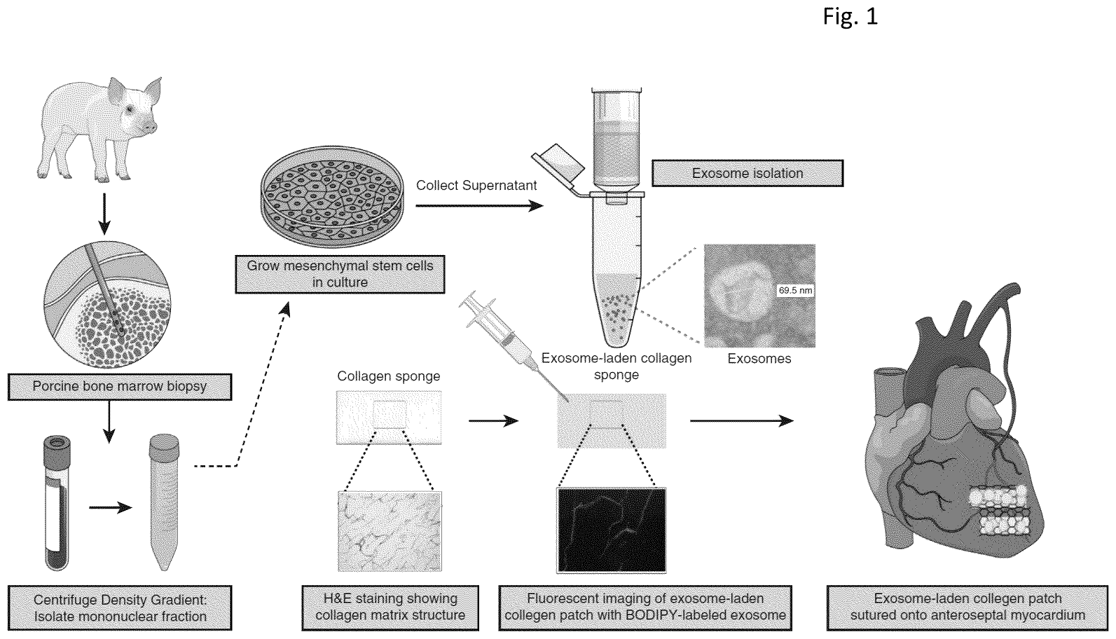

EXP was created using Helistat® hemostatic collagen sponge (Dental Implant Technologies, Scottsdale, AZ) embedded with purified exosomes. Purified exosomes (3 ml) injected into two 1.27 cm×2.54 cm (3.2 cm²) collagen sponges to create one EXP. Final patch holds approximately 3×10⁸ exosomes (FIG. 1). In vitro studies demonstrate that EXPs had sustained release of exosomes over 7 days (FIG. 9B). The collagen sponge was immersed in 1 ml PBS at 37° C. PBS was replaced every two days for first 7 days and one final time on day 8. Release was quantified by NanoSight as described above. We confirmed presence of exosomes in patch by fluorescence imaging and BODIPY-labeled exosomes followed the pattern of collagen matrix in EXP (FIGS. 9 C&D). EXP was placed on epicardial surface of hibernating region following CABG (FIG. 9E).

### Animal Use:

This study was approved by Institutional Animal Care and Use Committees of the Minneapolis VA Medical Center and University of Minnesota and was performed conforming to National Institutes of Health guidelines. A priori power analyses using G*Power software (Heinrich Heine University, Dusseldorf, Germany) were used to determine group sizes.

### Animal Study Design:

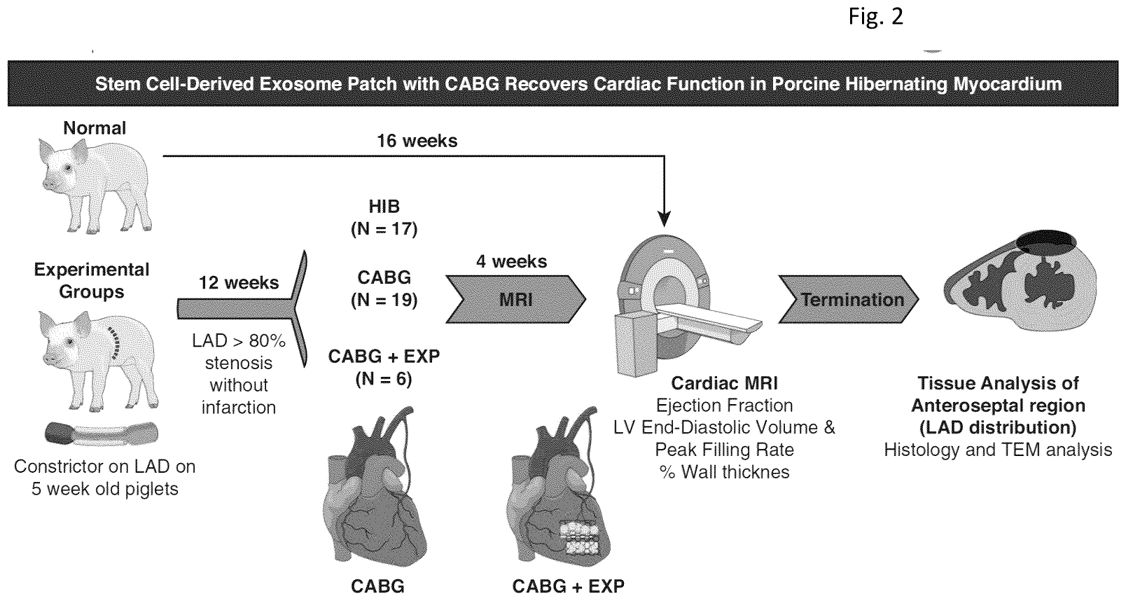

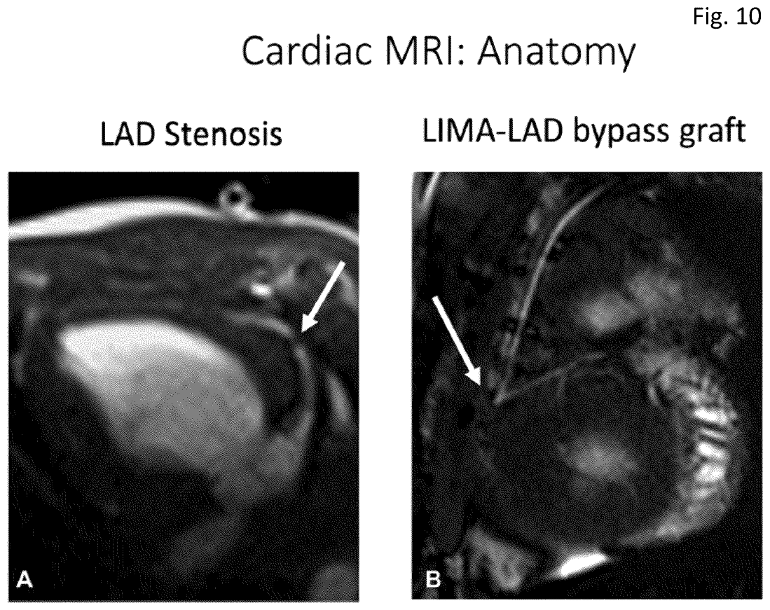

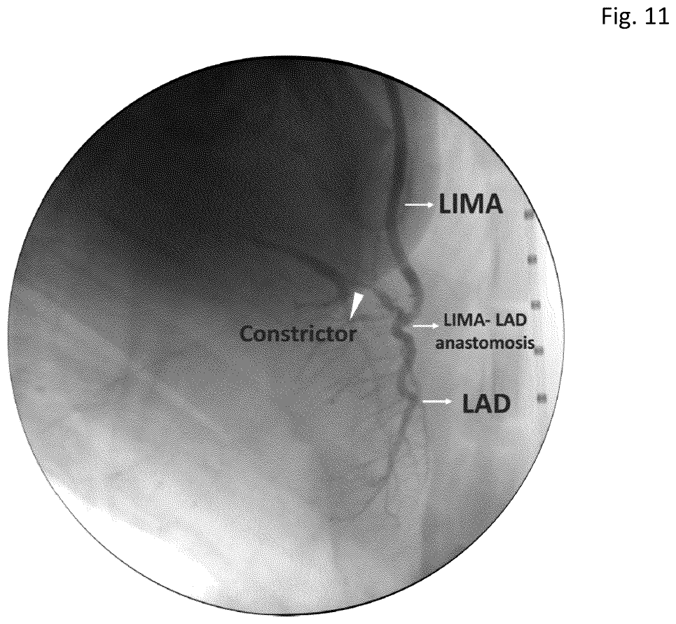

48 Yorkshire-Landrace juvenile swine were studied with groups of Normal (n=6), HIB (n=17), CABG (n=19), CABG+EXP (exosome laden collagen patch) (n=6). Only female juvenile swine were used in the study and matched for age, diet, and weight and the groups were non-randomized. Of 48 animals, 42 underwent hibernation procedure to induce 80% LAD stenosis cause chronic ischemia without infarction over 12 weeks. Subsequently, animals were assigned to termination or revascularization with or without EXP. CABG was performed using left internal mammary artery (LIMA) to LAD and collagen patch embedded with exosomes was applied to hibernating region in CABG+EXP group. CABG and CABG+EXP groups recovered for 4 weeks. Prior to termination, animals underwent cardiac MRI and angiography to assess global and regional function, diastolic relaxation, coronary anatomy, and tissue viability (FIG. 10, 11). Myocardial tissue was obtained for histologic and proinflammatory biomarker studies (FIG. 2).

### Swine Model of HIB:

Briefly, Juvenile swine were sedated, intubated, and anesthetized. Using a left thoracotomy approach, a plastic c-shaped constrictor with an internal diameter of 1.5 mm was placed on the LAD proximal to the first diagonal without occluding the artery and secured with non-absorbable sutures. This constrictor is not occlusive but creates stenosis as the animal grows. Animals recovered for 12 weeks, which is enough time to fully establish HIB phenotype with chronic myocardial ischemia without infarction in the LAD distribution.

Juvenile swine were sedated with tiletamine/zolazepam (4 mg/kg intramuscularly (IM)) and xylazine (2 mg/kg IM), intubated, and anesthetized with inhaled isoflurane (2%). Using a left thoracotomy approach, a plastic c-shaped constrictor with an internal diameter of 1.5 mm was placed on the LAD proximal to the first diagonal without occluding the artery and secured with non-absorbable sutures. This constrictor is not occlusive but creates stenosis as the animal grows. Animals recovered for 12 weeks, which is enough time to fully establish HIB phenotype with chronic myocardial ischemia without infarction in the LAD distribution. Proximal LAD stenosis was documented by CMRI, and myocardial viability was confirmed by normal gadolinium enhancement^{23,E18} Perfusion, global and regional function, wall motion strain and diastolic relaxation were measured.

### Off-Pump CABG+Exosome Laden Collagen Patch (EXP) Treatment:

The swine model of off-pump CABG has been previously well-characterized²³. Refer to Aggarwal et al for detailed methods.⁷ Briefly, 12 weeks after hibernation, pigs undergo CABG via median sternotomy with the LIMA pedicle graft dissected free from the chest wall. Lidocaine (1 mg/kg) and heparin (200 units/kg) was administered. The LAD artery distal to the site of stenosis was exposed, and arteriotomy was made to prepare for anastomosis. Using off-pump technique and coronary shunt, LIMA-LAD anastomosis was performed using 7-0 prolene sutures. Immediately following anastomosis, EXP was applied to epicardial surface of hibernating region, and vicryl patch was sewn over EXP with polypropylene 7-0 suture to secure it. Sternotomy was closed. Animals recovered for 4 weeks prior to sacrifice.

At the end of 12 weeks after hibernation, pigs undergo CABG under general anesthesia. CABG was performed via median sternotomy with the left internal mammary artery (LIMA) pedicle graft dissected free from the chest wall. Lidocaine (1 mg/kg) and heparin (200 units/kg) was administered. The left anterior descending (LAD) artery distal to the site of stenosis was exposed, and arteriotomy was made to prepare for anastomosis as well as to confirm arterial flow distal to stenosis, confirming the hibernation model of partial occlusion. Using off-pump technique and coronary shunt, LIMA-LAD anastomosis was performed using 7-0 prolene sutures.

### Cardiac MRI:

Cardiac MRI was performed to confirm >80% LAD stenosis and assess global and regional contractile function and diastolic relaxation. Viability imaging was performed following completion of stress imaging to confirm lack of myocardial infarction in LAD territory.

Cardiac MRI (CMRI) was performed to confirm >80% LAD stenosis and assess global and regional contractile function as well as diastolic function. Two CMRIs were performed—first was done 12 weeks after placement of constrictor band to assess for hibernation physiology and second was done 4 weeks after revascularization and placement of exosome patch to assess for improvement in cardiac function. CMRI was performed on a 1.5-Tesla clinical scanner with the animals under general anesthesia. Following the acquisition of localizers, breath-held retrospectively triggered steady-state free precession cine images were collected in parallel short-axis planes from the base to apex of the left ventricle to assess global function and peak LV filling rate. This protocol was acquired during rest and repeated during dobutamine challenge using 5 μg/kg/min to simulate stress. Delayed enhancement images were acquired 10 minutes after injecting gadolinium-based contrast agent gadobenate dimeglumine (Bracco Diagnostics Inc) to rule out infarction of the LAD territory. Remote segments of the left ventricle (LV) are defined as the posterolateral segments of the LV wall opposite the LAD distribution. Measurements including, global function, diastolic filling (measured by peak filling rate (PFR) normalized to end-diastolic volume (EDV), systolic function (measured by ejection fraction and wall thickness) were analyzed using CVI42 (Circle Cardiovascular Imaging Inc, Calgary, AB, Canada). To limit the risk of bias, the MRIs were interpreted by blinded specialists.

### Terminal Procedure:

Prior to terminal procedure, invasive hemodynamic monitoring was done using the flow directed catheter to measure left ventricle end diastolic volume and pressure both at rest and under low dose dobutamine infusion. The catheter was placed through an introducer in femoral artery that travels from the aorta into left ventricle.

Four weeks after revascularization, a second cardiac MRI was performed followed by sacrifice. Briefly, pigs were anesthetized using general endotracheal anesthesia. Again, redo sternotomy was performed. Adhesions were dissected around the LIMA-LAD graft with extreme caution. Heart was then explanted. Coronary dilators were used to assess the degree of LAD stenosis and measure the vessel diameter proximal and distal to the bypass graft. All animals had a patent graft measuring 2.5-3.5 mm. Tissue was obtained from anteroseptal (ischemic region of LAD territory) distal to the stenosis and graft anastomosis as well as from the lateral left ventricular wall (remote region) to assess for the differences in inflammation.

### Histologic Fibrosis and Inflammation Scoring:

Trichrome and alpha-smooth muscle actin (SMA) staining was used to demonstrate collagen infiltration and presence of myofibroblasts respectively into different layers of connective tissue in representative samples from LAD regions in HIB, CABG, CABG+EXP groups and compared to normal.

Myocardial samples from ischemic (anteroseptal) and non-ischemic (remote zone) regions were immediately rinsed and fixed in neutral buffered formalin, before being embedded in paraffin and stained with hematoxylin and eosin (H&E), trichrome and alpha-smooth muscle actin (SMA) staining for analysis of tissue structure, viability, and collagen deposits respectively. Briefly, a scoring system was developed by an experienced animal pathologist to quantify fibrosis and inflammation among the study groups (Table 1). Fibrosis grading was based on the location of fibrosis and inflammation scoring was based on the number of inflammatory cells seen per high power field. Measurements of areas were made using a calibrated combined microscope-camera-software system (Nikon Eclipse Ci microscope, Nikon digital Sight D5-U3 camera, NIS Elements D software). To limit risk of bias, all histological analyses were performed by a blinded animal pathologist.

TABLE 1
Histopathological scoring of fibrosis and inflammation
in a swine model of hibernating myocardium

| Grade of | Interstitial | Scoring of | Severity of |
|---|---|---|---|
| Fibrosis | Fibrosis | Inflammation | Inflammation |
| 1 | Limited to | A | No inflammatory |
| | perimysium | | response |
| 2 | Focal | B | 1-5 cells |
| | extension into | | per high- |
| | endomysium | | powered field |
| 3 | Multiple | C | 5-10 cells |
| | extension into | | per high- |
| | endomysium | | powered field |
| 4 | Extensive | D | 11-20 cells- |
| | presence into | | per high |
| | endomysium | | powered field |
| 5 | Replacement | E | >20 cells |
| | fibrosis | | per high- |
| | | | powered field |

### Transmission Electron Microscopy (TEM):

Myocardial tissue samples were obtained from LAD-supplied territory and fixed in 3% paraformaldehyde, 1.5% glutaraldehyde and 2.5% sucrose in 0.1 M sodium cacodylate buffer with 5 mM calcium chloride and 5 mM magnesium chloride (pH 7.4) for at least 24 hours at 4° C. before processing for electron microscopy.

Myocardial tissue samples were obtained from the LAD-supplied territory and fixed in 3% paraformaldehyde, 1.5% glutaraldehyde and 2.5% sucrose in 0.1 M sodium cacodylate buffer with 5 mM calcium chloride and 5 mM magnesium chloride (pH 7.4) for at least 24 hours at 4° C. They were subsequently rinsed in buffer and placed in 1% osmium tetroxide and 0.1 M sodium cacodylate buffer for 2 hours. Specimens were then rinsed in ultrapure water (NANOpure Infinity®; Barnstead/Thermo Fisher Scientific; Waltham, Maryland), stained with 2% aqueous uranyl acetate, and rinsed again. Following dehydration in ethanol they were embedded in Embed 812 resin (Electron Microscopy Sciences, Hatfield, Pennsylvania). Ultrathin sections 80-100 nm thick were cut on a Leica Ultracut UCT microtome using a diamond knife, collected on formvar/carbon-coated 2×1 mm slot grids (Electron Microscopy Sciences, Hatfield, Pennsylvania), and stained with 3% uranyl acetate, followed by Sato's triple-lead stain^{E16}. Sections were examined with a JEOL JEM-1400Plus transmission electron microscope operating at 60 kV. Images were recorded with an Advanced Microscopy Techniques XR16 camera using AMT Capture Engine software.

### Measurement of Mitochondrial Area:

Three animals were included per group with total of approximately 500 mitochondria measured in each group. TEM images were analyzed using Fiji image analysis tool²⁴. All images with correct magnification (4/8.8 k) were used. For all mitochondria, area, perimeter, major and minor axis were measured. Mitochondrial aspect ratios were calculated (aspect ratio=major axis/minor axis). Significant outliers (p<0.05) for measurements of each group were excluded. All measurements for animals of each group were combined and compared to CABG+EXP using one-way ANOVA followed by Tukey's test.

### Labeling and Fluorescent Imaging of Isolated Exosomes

Labeling procedures were carried out in accordance to a standard labeling protocol with modifications (Invitrogen, Carlsbad, USA). Briefly, isolated exosomes were labeled with 1 μl BODIPY TR ceramide stock solution (Invitrogen, Carlsbad, USA) per 100 μl sample in PBS for 20 minutes at 37° C. Excess BODIPY was removed using exosome-spin columns (Thermo Scientific, Schwerte, Germany). Labeled exosomes were then incorporated into the collagen patch in preparation for fluorescence imaging. Briefly, 500 μl of sample was injected on the collagen sponge, which was then placed in 2 ml 10% formalin for 48 hours for fixation. The sponge was then covered with coverslip. Fluorescence images were taken using the Olympus IX81 microscope (Tokyo, Japan). All images were post-processed and quantified using ImageJ.

### Statistics:

A priori power analyses using G*Power software (Heinrich Heine University, Dusseldorf, Germany) were used to determine animal group sizes. Statistical differences between groups were determined by using one-way analysis of variance (ANOVA) followed by Tukey's post hoc test as we were interested in computing confidence intervals for every comparison among our four groups. Where non-normality was determined with Shapiro-Wilk testing, data was analyzed using the Kruskal-Wallis test. Statistics were calculated using GraphPad PRISM 9.0 software (GraphPad Software, Inc, La Jolla, Calif). Probability values <0.05 were considered significant. The p-values for miRNA profiling and differential expression were adjusted using the Benjamini & Hoschberg method. Corrected p-value of 0.05 was set as threshold for significantly differential expression by default. Data are presented as means±SD or as means±SEM.

### Lyophilization Method:

Human bone-marrow-derived MSCs (hMSCs) were purchased from Ossium Health. To mimic hibernation conditions in vitro, AC16 human cardiomyocytes were exposed to mild hypoxia (1% 02 for 24 hours). The conditioned media from hypoxic AC16 cells was collected. hMSCs were cultured in hypoxic AC16 conditioned for 6 hours. Media was collected from hMSCs and centrifuged to remove debris and cells and concentrated using a 100 kDa molecular weight cut off Amicon Ultra centrifugal filter, and trehalose was added to a final concentration of 0.47 mM. Concentrated media containing extracellular vesicles was frozen at −80° C. and lyophilized under a >0.01 m vacuum for 24 hours and can be stored for 6 months at room temperature.

Lyophilized extracellular vesicles are reconstituted in 1× phosphate buffered saline. Extracellular vesicle particles per ml was determined using nanoparticle tracking analysis. Reconstituted lyophilized extracellular vesicles were brought to a final concentrate of 1×10⁸ extracellular vesicles per ml and pipetted onto the scaffold.

### Results

### Mitochondrial Respiratory Function:

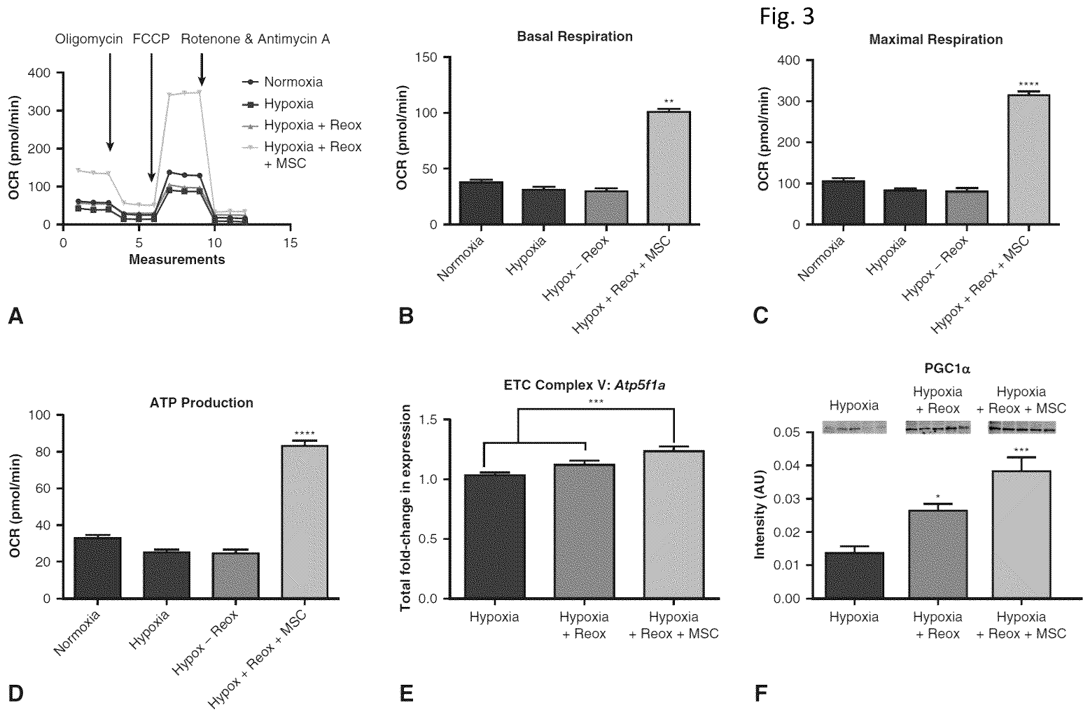

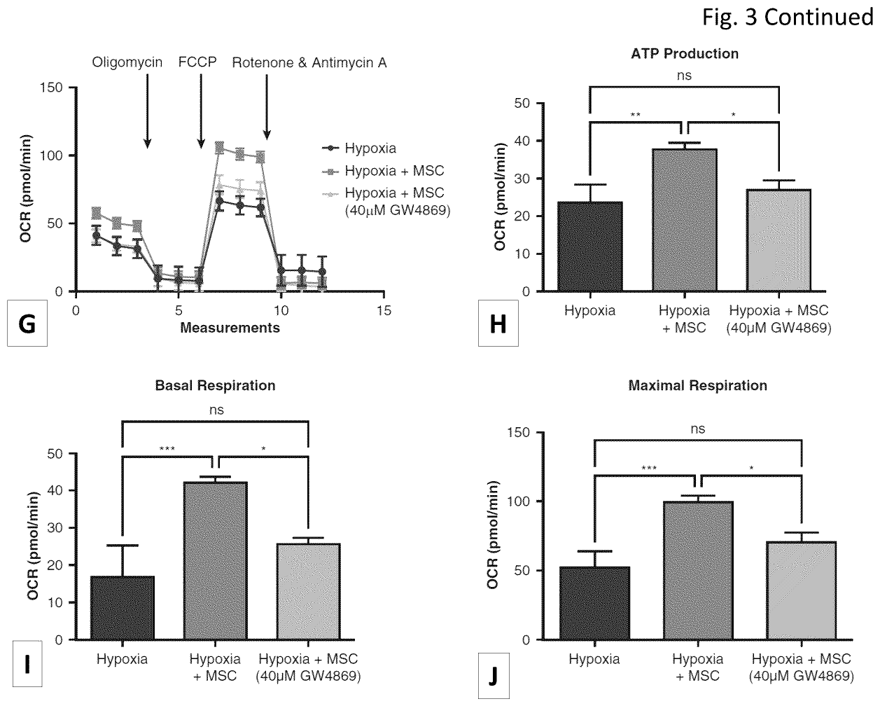

Our group has previously demonstrated co-culture of MSCs during reoxygenation restores mitochondrial respiratory function in hypoxic cardiomyocytes (FIG. 3 A-F)²⁵. We compared the expression of ETC. complex 5 by measuring the relative expression of Atp5f1a, which encodes the F1 alpha subunit of ETC complex 5. We found that Atp5f1a significantly increases in hypoxic H9C2 cells after reoxygenation and co-culture with MSCs. Pharmacological inhibition of exosome secretion from MSCs by GW4869 abrogated the positive increase in mitochondrial respiration. Measurement of oxygen consumption rate (OCR) in hypoxic cardiomyocytes following transwell co-culture with MSCs+GW4869 resulted in an overall decrease in mitochondrial respiratory activity compared to MSCs alone. Similarly, ATP production, basal respiration, and maximal respiration were decreased following co-culture with MSCs+GW4869 compared to MSCs alone (27±2.5 vs. 37.8±1.7, 25.7±1.7 vs. 42.1±1.7, 70.4±6.9 vs. 99.4±4.6 respectively; p<0.05) (FIG. 3 G-J).

### MicroRNA:

Using small RNA-sequencing, we identified eight differentially expressed miRNAs in exosomes released from MSCs in normoxia+reoxygenation H9C2 co-culture conditions versus exosomes released from MSCs in hypoxia+reoxygenation H9C2 co-culture conditions. While miR-21-5p, miR-10a-5p, and miR-143-3p were upregulated, miRNAs 126-3p, 92a, 151-3p, 486 and 423-5p were found to be downregulated in exosomes from hypoxia+reoxygenation H9C2 co-culture conditions (FIG. 4). Target prediction software predicted six miRNAs to target predefined genes of interest, including key regulators and components of mitochondria metabolism (PGC-1α, succinate dehydrogenase, ATP synthase) as well as proinflammatory mediators (NF-κB, IL-1β, IFNγ).

### Cardiac MRI Shows Improved Cardiac Function in CABG+EXP:

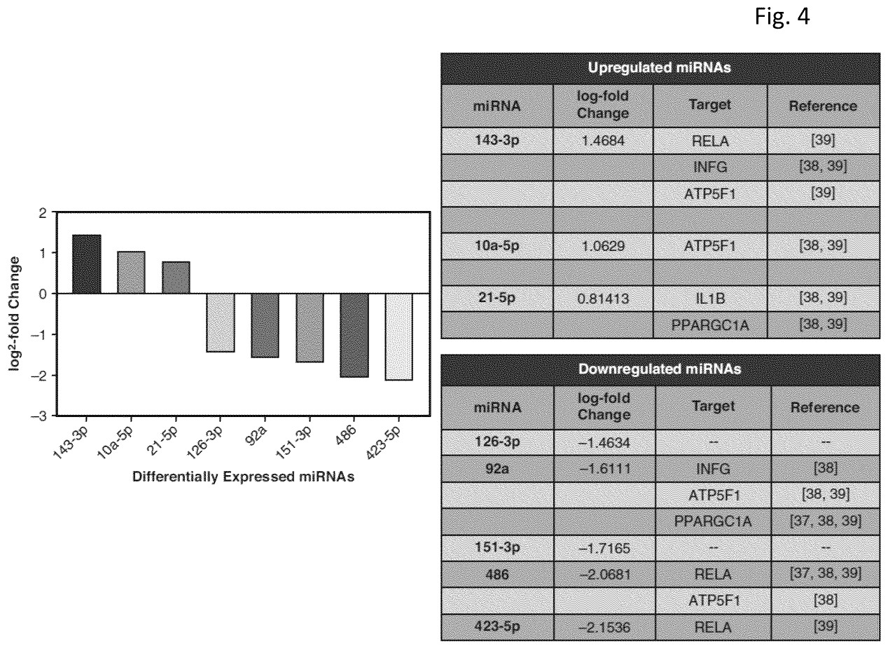

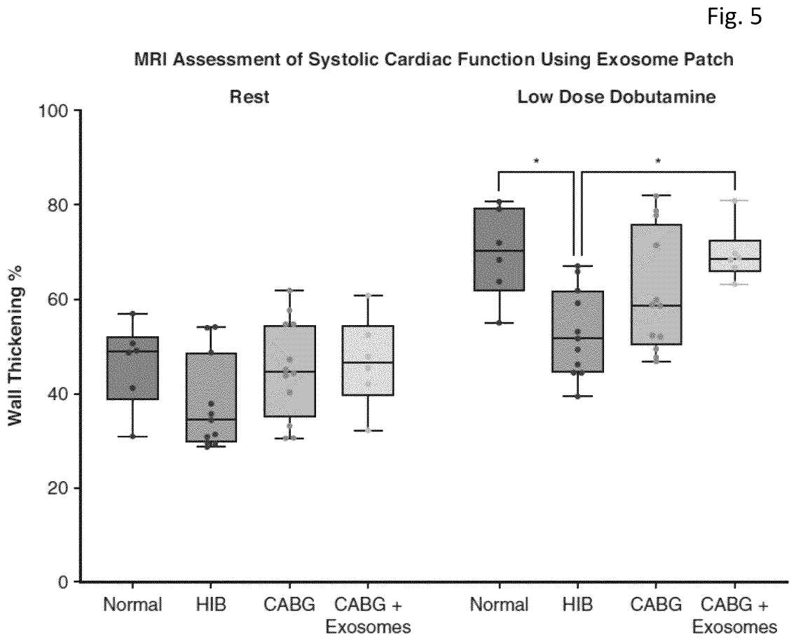

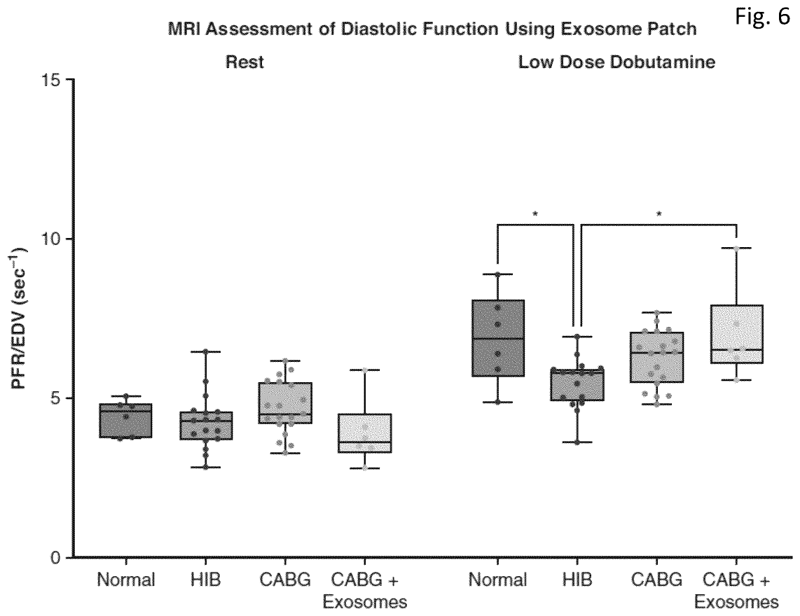

At rest, cardiac MRI showed preserved left ventricular ejection fraction (LVEF) among four groups (Normal, HIB, CABG, and CABG+EXP) (55.9±7.2%, 54.9±10.7%, 57.5±7.5%, 52.4±9.1%, respectively) with an increase under low dose dobutamine infusion at 5 μg/kg/min (74.2±11.2%, 65.7±11.1%, 69.9±8.1%, 67.2±7.6% respectively, p<0.01 for all groups). MRI analyses of regional systolic function, measured by circumferential wall thickening percentage, was similar among four groups at rest (46.24±9.02, 37.68±9.88, 45.32±10.51, 46.78±9.65 respectively). Under stress, there was decrease in circumferential wall thickening % in HIB compared to normal (52.93±9.33 vs. 69.80±9.69 respectively, p=0.02). In CABG, there was no significant improvement in wall thickening % compared to HIB (61.28±12.86 vs. 52.93±9.33 respectively, p=0.24). However, in CABG+EXP, there was improvement in wall thickening % when compared to HIB (69.6±5.99 vs. 52.93±9.33 respectively, p=0.02) (FIG. 5). At rest, peak filling rate/end diastolic volume (PFR/EDV) was similar among four groups (4.43±0.56, 4.26±0.88, 4.63±0.85, 3.92±1.05 respectively). Under stress, there was decrease in PFR/EDV in HIB compared to normal (5.49±0.77 vs. 6.88±1.43 respectively, p=0.03). In CABG, there was no significant improvement in PFR/EDV compared to HIB (6.25±0.87 vs. 5.49±0.77 respectively, p=0.07). However, in CABG+EXP, there was improvement in PFR/EDV when compared to HIB (6.99±1.43 vs. 5.49±0.77 respectively, p=0.02) (FIG. 6). Cardiac MRI showed >80% proximal LAD stenosis, absence of necrosis in HIB territory and patency of LIMA to LAD bypass graft distal to area of stenosis.

### Histologic Analysis:

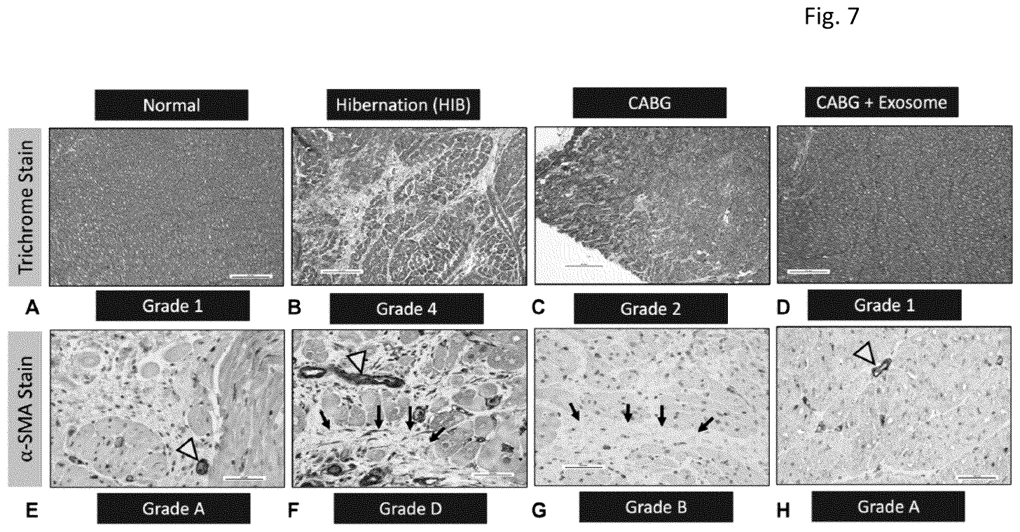

Normal group animals had score 1A, HIB group had score 4D, CABG group had scores 2B or 3C, and CABG+EXP group showed minimal fibrosis and inflammation with score 1A (FIG. 7 A-D). Presence of myofibroblasts was assessed using alpha SMA staining. Normal group showed absence of alpha-SMA stain in myocardial tissue beyond the expected smooth muscle in the microvasculature. The HIB group showed increased myofibroblasts in areas of fibrosis. The CABG showed very few myofibroblasts associated with alpha-SMA stain and there were no myofibroblasts seen in CABG+EXP (FIG. 7 E-H)

### TEM Analysis of Mitochondria Size and Structure:

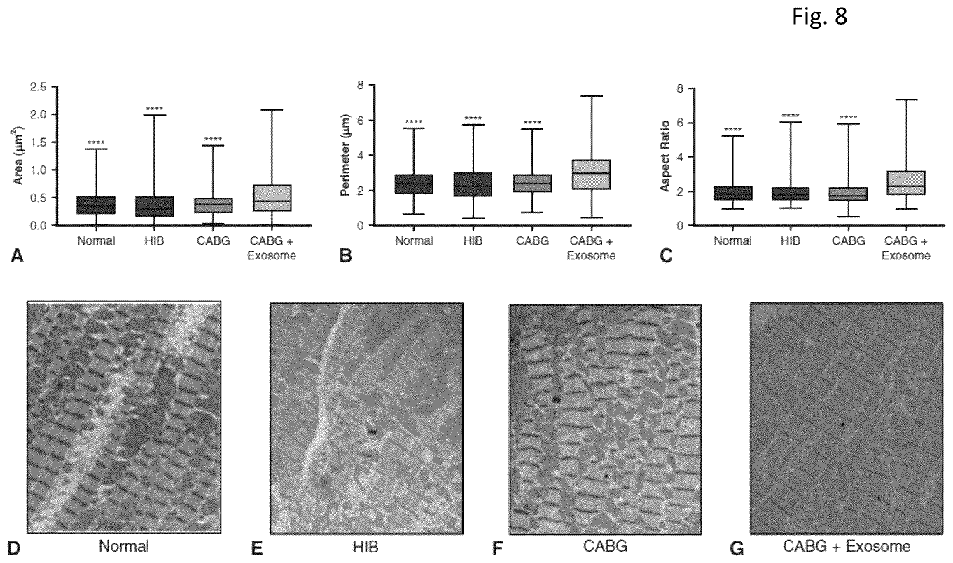

Compared to HIB or CABG alone, CABG+EXP showed increase in mitochondrial area (0.38±0.29, 0.40±0.23, 0.53±0.35 respectively; p<0.0001), perimeter (2.34±0.95, 2.45±0.82, 3.00±1.24 respectively; p<0.0001), and aspect ratio (1.98±0.75, 2.00±0.86, 2.63±1.12 respectively; p<0.0001) (FIG. 8 A-C). Representative TEM images (8800× magnification) of HIB show dysregulation of mitochondrial size and alignment which is recovered following treatment with exosomes (FIG. 8 D-G).

### Discussion

In this study, we investigated the role of MSC-derived exosomes with CABG to recover myocardial function in a region of chronic ischemia. We found exosomes delivered as an epicardial patch to the HIB region significantly improve cardiac function, mitochondrial morphology and metabolism, and myocardial fibrosis.

Our previous work with the porcine model of HIB showed that systolic and diastolic function and mitochondrial adaptations in HIB remain impaired despite CABG^{7,23,25}. A chronic persistence of metabolic and functional adaptations seen in HIB, even after restoration of sufficient blood supply, might explain remaining impairments in contractile function that we observed in our swine model. Adaptations in HIB tissue include downregulation of mitochondrial energy metabolism to preserve viability and reduce oxidant stress but results in lower production of ATP⁹. Cardiac tissue has high energetic demand, making it critically dependent on effective mitochondrial function. The recovery of mitochondrial metabolism, dynamics and biogenesis is crucial to restoring bioenergetics and contractile function in ischemic heart^{9,26}.

We have previously demonstrated inadequate mitochondrial bioenergetic recovery despite CABG, as manifested by persistently low expression of ETC proteins and PGC-1α, a key driver of mitochondrial biogenesis⁹. Using TEM, we have observed mitochondria remained smaller and more variable in size despite CABG, which could be explained by dysregulation of mitochondrial fusion and fission. We developed an in vitro model of HIB to allow for direct manipulation and measurement of mitochondrial respiration. We observed that addition of MSCs during reoxygenation restores mitochondrial respiratory function in hibernating cardiomyocytes, including an overall increase in basal and maximal respiration, ATP production, and ETC proteins²⁵. In the porcine model of HIB, adjunctive use of an MSC patch during CABG showed recovery in regional systolic and diastolic function, as well as increased expression of ETC complex II and V and PGC-1α suggesting that the mechanism by which MSCs improve cardiac function is mitochondrial-based via paracrine signaling^{17,27}.

While a primary goal of stem cells was to form de-novo cardiomyocytes²⁸, studies found they exert their benefits largely via their secretome^{29,30}. Specifically, exosomes secreted from stem cells have shown to attenuate ischemic injury in both small and large animal models^{15,16}. Exosomes are secreted stable microvesicles (50-150 nm diameter) that contain either protein or nucleic acids, including microRNA (miRNA)³¹. Exosomes have emerged as key signaling mechanism of cell-based regenerative therapies including enhancement of angiogenesis and cardiomyocyte proliferation³², improved cardiac function, attenuated proinflammatory signaling and reduced cardiomyocyte death³³. Importantly, they may regulate mitochondrial metabolism in the context of tissue hypoxia³⁴.

Various aspects of exosome-associated cardioprotective effects have been linked to miRNAs, which are contained and transported within the extracellular vesicles. miRNAs are key drivers of exosome-induced functional recovery and reduction in tissue scarring in response to myocardial ischemia³⁵. These miRNAs are critical because they can individually modulate multiple different processes leading to pleiotropic effects^{E1}. In this study, we identified differentially expressed miRNAs in exosomes, derived from MSCs after co-culture with hypoxic H9C2 cells. We found three miRNAs to be upregulated in response to ischemic paracrine signals: miR-143-3p, miR-10a-5p and miR-21-5p. miR-143 plays a key role in vascular smooth muscle cell differentiation and is secreted in response to vascular injury^{E2,E3}. In zebrafish, miR-143-add3 pathway has been shown to regulate cardiomyocyte morphology and function^{E4}. Furthermore, upregulation of miR-143 has been linked to the development of cardiomyopathy^{E5}. Similarly, miR-10a-5p has been shown to have vascular effects, by regulating endothelial inflammation. There is contradicting evidence regarding the effects of miR-10a on inflammatory signaling and its effects on cardiomyocytes^{E6,E7}. miR-21-5p has been implicated in heart failure, myocardial infarction, and cardiomyopathies. Similar to other miRNAs, such as miR-10a-5p, the exact effects on cardiomyocytes in heath and disease remain unclear. While some studies attribute cardioprotective properties to miR-21, other link it to cardiac fibrosis and contractile dysfunction^{E8-E11}. Using multiple target prediction tools, we found that all three upregulated miRNAs may interact with genes involved in mitochondria metabolism or inflammatory signaling, including PGC-1α and NF-κB.

This study focused on using MSC-derived exosomes in an ischemic heart disease model to improve mitochondrial bioenergetics and reduce myocardial fibrosis to restore normal myocardial function. No other groups have shown this relationship in a cell-free therapy model of HIB+CABG. Most literature regarding cell-free therapies has focused on infarcted models and mechanisms involving increasing neovascularization and inhibition of apoptosis rather than endpoints relevant to models of chronically ischemic myocardium. In this study, we have shown that exosomes exert many similar effects as MSCs, including recovery of cardiac function, improvement of mitochondrial biogenesis and reduction in inflammation. Compared to stem cells, exosomes are easy to isolate and can be stored for long periods of time, presenting an opportunity for off-the-shelf products that can be used in the acute setting making it more translatable into a clinical context. Furthermore, recent studies have suggested that exosomes have low immunogenicity compared to cell therapies, thereby avoiding the risk of detrimental recipient immunogenic responses^{E12}. A variety of other methods have been suggested for delivering MSCs or exosomes into the heart such as intramyocardial injection, however, this results in minimal concentrations of therapeutic product in injured area, with up to 90% of cells dying or washing away in cell injection^{E13}. Analysis of exosomes retention following injection has been completed for up to 3 hours post injection and has shown significant decreases in the exosome content^{E14}. Our preliminary data confirmed presence of exosomes in patch by fluorescence imaging and sustained release of exosomes over a week. As opposed to hydrogels, this exosome-laden patch can be surgically secured to ischemic region, which allows for gradual release of exosomes over a week resulting in continuous and direct signaling to ischemic region that has been revascularized.

This study demonstrates recovery of myocardial systolic and diastolic function in porcine model of chronically ischemic myocardium when EXP is applied during CABG. Histologic and molecular studies showed reduced fibrosis and inflammatory signaling, as well as increased mitochondrial size and aspect ratio. Further analyses suggest miRNAs are an important signaling mechanism in regenerative effects of exosomes.

By improving our understanding on the mechanisms underlying exosome-driven restoration of cardiac function in HIB, we pave the way for applications of an off-the-shelf cell-free adjunctive therapy to be used alongside surgical revascularization to improve clinical outcomes in coronary artery disease.

### REFERENCES

1. Dai H, Much A A, Maor E, et al. Global, regional, and national burden of ischaemic heart disease and its attributable risk factors, 1990-2017: results from the Global Burden of Disease Study 2017. Eur Heart J Qual Care Clin Outcomes. Jan. 5 2022; 8(1):50-60. doi:10.1093/ehjqcco/qcaa076
2. Tsao C W, Aday A W, Almarzooq Z I, et al. Heart Disease and Stroke Statistics-2022 Update: A Report From the American Heart Association. Circulation. Feb. 22 2022; 145(8):e153-e639. doi:10.1161/CIR.0000000000001052
3. Rahimtoola S H. The hibernating myocardium. Am Heart J. January 1989; 117(1):211-21. doi:10.1016/0002-8703(89)90685-6
4. Kukulski T, She L, Racine N, et al. Implication of right ventricular dysfunction on long-term outcome in patients with ischemic cardiomyopathy undergoing coronary artery bypass grafting with or without surgical ventricular reconstruction. J Thorac Cardiovasc Surg. May 2015; 149(5):1312-21. doi:10.1016/j.jtcvs.2014.09.117
5. Kelly R F, Cabrera J A, Ziemba E A, et al. Continued depression of maximal oxygen consumption and mitochondrial proteomic expression despite successful coronary artery bypass grafting in a swine model of hibernation. J Thorac Cardiovasc Surg. January 2011; 141(1):261-8. doi:10.1016/j.jtcvs.2010.08.061
6. Hocum Stone L, Wright C, Chappuis E, et al. Surgical Swine Model of Chronic Cardiac Ischemia Treated by Off-Pump Coronary Artery Bypass Graft Surgery. J Vis Exp. Mar. 27 2018; (133)doi:10.3791/57229
7. Aggarwal R, Qi S S, So S W, et al. Persistent diastolic dysfunction in chronically ischemic hearts following coronary artery bypass graft. J Thorac Cardiovasc Surg. Aug. 24 2022; doi:10.1016/j.jtcvs.2022.08.010
8. Cabrera J A, Butterick T A, Long E K, et al. Reduced expression of mitochondrial electron transport chain proteins from hibernating hearts relative to ischemic preconditioned hearts in the second window of protection. J Mol Cell Cardiol. July 2013; 60:90-6. doi:10.1016/j.yjmcc.2013.03.018
9. Holley C T, Long E K, Butterick T A, et al. Mitochondrial fusion proteins in revascularized hibernating hearts. J Surg Res. May 1 2015; 195(1):29-36. doi:10.1016/j.jss.2014.12.052
10. Vidoni S, Zanna C, Rugolo M, Sarzi E, Lenaers G. Why mitochondria must fuse to maintain their genome integrity. Antioxid Redox Signal. Aug. 1 2013; 19(4):379-88. doi:10.1089/ars.2012.4800
11. Bartunek J, Terzic A, Davison B A, et al. Cardiopoietic cell therapy for advanced ischaemic heart failure: results at 39 weeks of the prospective, randomized, double blind, sham-controlled CHART-1 clinical trial. Eur Heart J. March 12017; 38(9):648-660. doi:10.1093/eurheartj/ehw543
12. Karbasiafshar C, Sellke F W, Abid M R. Mesenchymal stem cell-derived extracellular vesicles in the failing heart: past, present, and future. Am J Physiol Heart Circ Physiol. May 1 2021; 320(5):H1999-H2010. doi:10.1152/ajpheart.00951.2020
13. Wagner M J, Khan M, Mohsin S. Healing the Broken Heart; The Immunomodulatory Effects of Stem Cell Therapy. Front Immunol. 2020; 11:639. doi:10.3389/fimmu.2020.00639
14. Aggarwal R, Potel K N, Shao A, et al. An Adjuvant Stem Cell Patch with Coronary Artery Bypass Graft Surgery Improves Diastolic Recovery in Porcine Hibernating Myocardium. Int J Mol Sci. Mar. 13 2023; 24(6)doi:10.3390/ijms24065475
15. Khan M, Nickoloff E, Abramova T, et al. Embryonic stem cell-derived exosomes promote endogenous repair mechanisms and enhance cardiac function following myocardial infarction. Circ Res. Jun. 19 2015; 117(1):52-64. doi:10.1161/CIRCRESAHA.117.305990
16. Bian S, Zhang L, Duan L, Wang X, Min Y, Yu H. Extracellular vesicles derived from human bone marrow mesenchymal stem cells promote angiogenesis in a rat myocardial infarction model. J Mol Med (Berl). April 2014; 92(4):387-97. doi:10.1007/s00109-013-1110-5
17. Hocum Stone L L, Swingen C, Wright C, et al. Recovery of hibernating myocardium using stem cell patch with coronary bypass surgery. J Thorac Cardiovasc Surg. July 2021; 162(1):e3-e16. doi:10.1016/j.jtcvs.2019.12.073
18. Salvatori I, Ferri A, Scaricamazza S, et al. Differential toxicity of TAR DNA-binding protein 43 isoforms depends on their submitochondrial localization in neuronal cells. J Neurochem. September 2018; 146(5):585-597. doi:10.1111/jnc.14465
19. Zhou L, Chen J, Li Z, et al. Integrated profiling of microRNAs and mRNAs: microRNAs located on Xq27.3 associate with clear cell renal cell carcinoma. PLoS One. Dec. 30 2010; 5(12):e15224. doi:10.1371/journal.pone.0015224
20. Chen Y, Wang X. miRDB: an online database for prediction of functional microRNA targets. Nucleic Acids Res. Jan. 8 2020; 48(D1):D127-D131. doi:10.1093/nar/gkz757
21. Vejnar C E, Blum M, Zdobnov E M. miRmap web: Comprehensive microRNA target prediction online. Nucleic Acids Res. July 2013; 41(Web Server issue):W165-8. doi:10.1093/nar/gkt430
22. Lu T P, Lee C Y, Tsai M H, et al. miRSystem: an integrated system for characterizing enriched functions and pathways of microRNA targets. PLoS One. 2012; 7(8):e42390. doi:10.1371/journal.pone.0042390
23. Hocum Stone L L, Swingen C, Holley C, et al. Magnetic resonance imaging assessment of cardiac function in a swine model of hibernating myocardium 3 months following bypass surgery. J Thorac Cardiovasc Surg. March 2017; 153(3):582-590. doi:10.1016/j.jtcvs.2016.10.089
24. Schindelin J, Arganda-Carreras I, Frise E, et al. Fiji: an open-source platform for biological-image analysis. Nat Methods. Jun. 28 2012; 9(7):676-82. doi:10.1038/nmeth.2019
25. Stone L L H, Chappuis E, Marquez M, McFalls E O, Kelly R F, Butterick T. Mitochondrial Respiratory Capacity is Restored in Hibernating Cardiomyocytes Following Co-Culture with Mesenchymal Stem Cells. Cell Med. 2019; 11:2155179019834938. doi:10.1177/2155179019834938
26. Chen Y, Liu Y, Dorn G W, 2nd. Mitochondrial fusion is essential for organelle function and cardiac homeostasis. Circ Res. Dec. 9 2011; 109(12):1327-31. doi:10.1161/CIRCRESAHA.111.258723
27. Aggarwal R, Potel K N, Shao A, et al. An Adjuvant Stem Cell Patch with Coronary Artery Bypass Graft Surgery Improves Diastolic Recovery in Porcine Hibernating Myocardium. International Journal of Molecular Sciences. 2023; 24(6):5475.
28. Toma C, Pittenger M F, Cahill K S, Byrne B J, Kessler P D. Human mesenchymal stem cells differentiate to a cardiomyocyte phenotype in the adult murine heart. Circulation. Jan. 1 2002; 105(1):93-8. doi:10.1161/hc0102.101442
29. Karantalis V, Hare J M. Use of mesenchymal stem cells for therapy of cardiac disease. Circ Res. Apr. 10 2015; 116(8):1413-30. doi:10.1161/CIRCRESAHA.116.303614
30. Godier-Furnemont A F, Martens T P, Koeckert M S, et al. Composite scaffold provides a cell delivery platform for cardiovascular repair. Proc Natl Acad Sci USA. May 10 2011; 108(19):7974-9. doi:10.1073/pnas.1104619108
31. Henning R J. Cardiovascular Exosomes and MicroRNAs in Cardiovascular Physiology and Pathophysiology. J Cardiovasc Transl Res. April 2021; 14(2):195-212. doi:10.1007/s12265-020-10040-5
32. Ibrahim A G, Cheng K, Marban E. Exosomes as critical agents of cardiac regeneration triggered by cell therapy. Stem Cell Reports. May 6 2014; 2(5):606-19. doi:10.1016/j.stemer.2014.04.006
33. Zheng Y L, Wang W D, Cai P Y, et al. Stem cell-derived exosomes in the treatment of acute myocardial infarction in preclinical animal models: a meta-analysis of randomized controlled trials. Stem Cell Res Ther. Apr. 8 2022; 13(1):151. doi:10.1186/s13287-022-02833-z
34. Zhang Y, Tan J, Miao Y, Zhang Q. The effect of extracellular vesicles on the regulation of mitochondria under hypoxia. Cell Death Dis. Apr. 6 2021; 12(4):358. doi:10.1038/s41419-021-03640-9
35. Sanchez-Sanchez R, Gomez-Ferrer M, Reinal I, et al. miR-4732-3p in Extracellular Vesicles From Mesenchymal Stromal Cells Is Cardioprotective During Myocardial Ischemia. Front Cell Dev Biol. 2021; 9:734143. doi:10.3389/fcell.2021.734143

### ADDITIONAL REFERENCES

E1 Olson E N. MicroRNAs as therapeutic targets and biomarkers of cardiovascular disease. Sci Transl Med. Jun. 4 2014; 6(239):239ps3. doi:10.1126/scitranslmed.3009008
E2 Rangrez A Y, Massy Z A, Metzinger-Le Meuth V, Metzinger L. miR-143 and miR-145: molecular keys to switch the phenotype of vascular smooth muscle cells. Circ Cardiovasc Genet. April 2011; 4(2):197-205. doi:10.1161/CIRCGENETICS.110.958702
E3 Zhao W, Zhao S P, Zhao Y H. MicroRNA-143/-145 in Cardiovascular Diseases. Biomed Res Int. 2015; 2015:531740. doi:10.1155/2015/531740
E4 Deacon D C, Nevis K R, Cashman T J, et al. The miR-143-adducin3 pathway is essential for cardiac chamber morphogenesis. Development. June 2010; 137(11):1887-96. doi:10.1242/dev.050526
E5 Ogawa K, Noda A, Ueda J, et al. Forced expression of miR-143 and -145 in cardiomyocytes induces cardiomyopathy with a reductive redox shift. Cell Mol Biol Lett. 2020; 25:40. doi:10.1186/s11658-020-00232-x
E6 Fang Y, Shi C, Manduchi E, Civelek M, Davies P F. MicroRNA-10a regulation of proinflammatory phenotype in athero-susceptible endothelium in vivo and in vitro. Proc Natl Acad Sci USA. Jul. 27 2010; 107(30):13450-5. doi:10.1073/pnas.1002120107
E7 Li P F, He R H, Shi S B, et al. Modulation of miR-10a-mediated TGF-beta1/Smads signaling affects atrial fibrillation-induced cardiac fibrosis and cardiac fibroblast proliferation. Biosci Rep. Feb. 28 2019; 39(2)doi:10.1042/BSR20181931
E8 Surina S, Fontanella R A, Scisciola L, Marfella R, Paolisso G, Barbieri M. miR-21 in Human Cardiomyopathies. Front Cardiovasc Med. 2021; 8:767064. doi:10.3389/fcvm.2021.767064
E9 Zhang J, Xing Q, Zhou X, et al. Circulating miRNA21 is a promising biomarker for heart failure. Mol Med Rep. November 2017; 16(5):7766-7774. doi:10.3892/mmr.2017.7575
E10 Ramanujam D, Schon A P, Beck C, et al. MicroRNA-21-Dependent Macrophage-to-Fibroblast Signaling Determines the Cardiac Response to Pressure Overload. Circulation. Apr. 13 2021; 143(15):1513-1525. doi:10.1161/CIRCULATIONAHA.120.050682
E11 Li Y, Chen X, Jin R, et al. Injectable hydrogel with MSNs/microRNA-21-5p delivery enables both immunomodification and enhanced angiogenesis for myocardial infarction therapy in pigs. Sci Adv. 2021; 7(9):eabd6740. doi:10.1126/sciadv.abd6740
E12 Zhu X, Badawi M, Pomeroy S, et al. Comprehensive toxicity and immunogenicity studies reveal minimal effects in mice following sustained dosing of extracellular vesicles derived from HEK293T cells. J Extracell Vesicles. 2017; 6(1):1324730. doi:10.1080/20013078.2017.1324730
E13 Hou D, Youssef E A, Brinton T J, et al. Radiolabeled cell distribution after intramyocardial, intracoronary, and interstitial retrograde coronary venous delivery: implications for current clinical trials. Circulation. Aug. 30 2005; 112(9 Suppl):I150-6. doi:10.1161/CIRCULATIONAHA.104.526749
E14 Gallet R, Dawkins J, Valle J, et al. Exosomes secreted by cardiosphere-derived cells reduce scarring, attenuate adverse remodelling, and improve function in acute and chronic porcine myocardial infarction. Eur Heart J. Jan. 14 2017; 38(3):201-211. doi:10.1093/eurheartj/ehw240
E15 Wang Q L, Wang H J, Li Z H, Wang Y L, Wu X P, Tan Y Z. Mesenchymal stem cell-loaded cardiac patch promotes epicardial activation and repair of the infarcted myocardium. J Cell Mol Med. September 2017; 21(9):1751-1766. doi:10.1111/jcmm.13097
E16 Sato T. A modified method for lead staining of thin sections. J Electron Microsc (Tokyo). 1968; 17(2):158-9.
E17 Xiao C, Wang K, Xu Y, et al. Transplanted Mesenchymal Stem Cells Reduce Autophagic Flux in Infarcted Hearts via the Exosomal Transfer of miR-125b. Circ Res. Aug. 17 2018; 123(5):564-578. doi:10.1161/CIRCRESAHA.118.312758
E18 Jerosch-Herold M, Swingen C, Seethamraju R T. Myocardial blood flow quantification with MRI by model-independent deconvolution. Med Phys. May 2002; 29(5):886-97. doi:10.1118/1.1473135

What is claimed:

1. A method of making an extracellular vesicle composition, the method comprising:
(a) contacting a mesenchymal stem cell (MSC) in vitro under normoxic conditions with (i) conditioned media from an ischemic cell or (ii) a cell exposed to hypoxic culture conditions for at least 12 hours prior to indirect co-culture with the MSC, to produce a contacted MSC culture, and
(b) collecting extracellular vesicles from the contacted MSC culture to generate the extracellular vesicle composition.

2. The method of claim 1, wherein conditioned media from the ischemic cell is generated by a method comprising: exposing a cell to hypoxic culture conditions for at least 12 hours and harvesting the culture media from the cells to create the conditioned media from the ischemic cell of step (a).

3. The method of claim 1, wherein the contacting of step (a) is for at least 6 hours.

4. The method of claim 1, wherein the ischemic cell or the cell exposed to hypoxic conditions are cardiomyocytes.

5. The method of claim 1, further comprising contacting a scaffold with the extracellular vesicle composition of step (b) to create an extracellular vesicle loaded scaffold, wherein the scaffold is made of an absorbable material and wherein the scaffold does not comprise intact cells.

6. The method of claim 5, wherein the scaffold is a hemostatic sponge, an absorbable gelatin sponge or an absorbable collagen sponge.

7. The method of claim 1, wherein the ischemic cell is a cardiomyocyte which has been exposed in vitro to hypoxic conditions for at least 12 hours, wherein the ischemic cardiomyocyte and the mesenchymal stem cell are indirectly co-cultured for at least 6 hours, and wherein the scaffold is a collagen sponge.

8. The method of claim 1, wherein hypoxic culture conditions comprise culturing cells in 0.5% to 10% oxygen for at least 12 hours.

9. The extracellular vesicle composition made by the method of claim 1.

10. A method of using the extracellular vesicle composition of claim 9, comprising administering the extracellular vesicle composition to a site of reduced blood supply in a subject.

11. The method of claim 10, wherein the extracellular vesicle composition is added to a scaffold wherein the scaffold is held in place at the site of reduced blood supply by a surgical mesh, wherein the surgical mesh maintains the position of the scaffold at the site of reduced blood supply.

12. The method of claim 10, wherein the subject has an ischemic injury.

13. The method of claim 12, wherein the ischemic injury comprises myocardial ischemia, mesenteric ischemia, peripheral ischemia, ischemic stroke, transient ischemic attack or cerebral ischemia.

14. The method of claim 10, wherein the subject is diagnosed with or undergoing surgery for coronary heart disease, a wound, a burn, organ transplant, stroke, severed limb, or a bone fracture.

15. A method of using the extracellular vesicle composition of claim 9, comprising administering the extracellular vesicle composition to a cardiac muscle of a subject, wherein the extracellular vesicle composition increases cardiac function.

16. A method of adjuvant vascular bypass therapy comprising:
a) culturing cardiomyocytes in hypoxic conditions for at least 12 hours to generate conditioned medium from an ischemic cell and ischemic cells;
b) culturing mesenchymal stem cells in normoxic conditions for at least 6 hours with one of (i) the ischemic cells of step (a) indirectly or (ii) the conditioned media from the ischemic cells;
c) collecting extracellular vesicles from the mesenchymal stem cells after the culturing of step (b); and
d) applying the extracellular vesicle to the site of a bypass graft.

17. The method of claim 16, wherein the bypass therapy is cardiac bypass, cerebral bypass or peripheral vascular bypass.

18. The method of claim 16, wherein the hypoxic conditions comprise 0.5% to 10% oxygen.

19. The method of claim 16, further comprising contacting the extracellular vesicle to an adsorbable scaffold to generate an extracellular vesicle loaded scaffold.

20. The method of claim 19, further comprising securing the extracellular vesicle-loaded scaffold to the site of the bypass with a surgical mesh.
